(19) United States
(12) Patent Application Publication
(10) Pub. No.: US 2022/0275097 A1
(43) Pub. Date: Sep. 1, 2022

(54) ANTIBODY TO LEPTIN RECEPTOR

(71) Applicant: SHANGHAITECH UNIVERSITY, Pudong District, Shanghai (CN)

(72) Inventors: Guang YANG, Shanghai (CN); Pingdong TAO, Shanghai (CN)

(73) Assignee: SHANGHAITECH UNIVERSITY, Pudong District, Shanghai (CN)

(21) Appl. No.: 17/623,792

(22) PCT Filed: Jul. 2, 2019
(86) PCT No.: PCT/CN2019/094352

§ 371 Date: Dec. 29, 2021

(51) Int. Cl. C07K 16/28; A61P 3/04

(52) U.S. Cl. CPC C07K 16/2869; A61P 3/04; C07K 2317/34; C07K 2317/565; C07K 2317/74; C07K 2317/75

(57) ABSTRACT

The present technology relates generally to compositions and methods of preventing or treating diseases associated with mutantleptin receptors, leptin deficiency or leptin dysfunction. The present technology also relates to administering the anti-leptin receptor antibodies in effective amounts to treat a subject suffering from, or predisposed to, a disease associated with mutant leptin receptors, obesity, leptin deficiency, leptin resistance, and/or hypoleptinemia.

### TECHNICAL FIELD

The present technology relates generally to immunoglobulin-related compositions (e.g., antibodies or antigen binding fragments thereof) that specifically bind leptin receptor protein and uses of the same. In particular, the present technology relates to the preparation of leptin receptor binding antibodies and their use in detecting and treating a disorder associated with or caused by leptin deficiency or hypoleptinemia, leptin resistance, and/or leptin receptor mutations causing defective or impaired leptin signaling, including obesity.

### BACKGROUND

The following description is provided to assist the understanding of the reader. None of the information provided or references cited is admitted to be prior art.

Obesity, including childhood obesity, is occurring at alarming rates around the world, with a prevalence of 12% globally. Obesity is also accompanied by high rates of serious, life-threatening, complications such as type 2 diabetes, cardiovascular disease and cancer. The underlying causes of obesity are complex, which include obesogenic environment and genetic susceptibility. Monogenic and syndromic obesity also exists.

### SUMMARY OF THE PRESENT DISCLOSURE

In one aspect, the present disclosureprovidesan anti-leptin receptor antibody, or antigen binding fragment thereof comprising a heavy chain immunoglobulin variable domain (V_{H}) and a light chain immunoglobulin variable domain (V_{L}), wherein the V_{H} comprises a V_{H}-CDR1 sequence selected from the group consisting of: SEQ ID NOs: 3, 13, 23, 33, 43, 53, 63, 73, and 83; a V_{H}-CDR2 sequence of selected from the group consisting of: SEQ ID NOs: 4, 14, 24, 34, 44, 54, 64, 74, and 84; and a V_{H}-CDR3 sequence selected from the group consisting of: SEQ ID NOs: 5, 15, 25, 35, 45, 55, 65, 75, and 85; and the V_{L} comprises an amino acid sequence selected from the group consisting of: a V_{L}-CDR1 sequence selected from the group consisting of: SEQ ID NOs: 8, 18, 28, 38, 48, 58, 68, 78, and 88; a V_{L}-CDR2 sequence of selected from the group consisting of: SEQ ID NOs: 9, 19, 29, 39, 49, 59, 69, 79, and 89; and a V_{H}-CDR3 sequence selected from the group consisting of: SEQ ID NOs: 10, 20, 30, 40, 50, 60, 70, 80, and 90.

In one aspect, the present disclosure provides an antibody or antigen binding fragment thereof comprising a V_{H} amino acid sequence comprising SEQ ID NO: 2, SEQ ID NO: 12, SEQ ID NO: 22, SEQ ID NO: 32, SEQ ID NO: 42, SEQ ID NO: 52, SEQ ID NO: 62, SEQ ID NO: 72, SEQ ID NO: 82, or a variant thereof having one or more conservative amino acid substitutions and/or a V_{L} amino acid sequence comprising SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87 or a variant thereof having one or more conservative amino acid substitutions.

Additionally or alternatively, in some embodiments, the antibody or antigen binding fragment comprises a V_{H} amino acid sequence and a V_{L} amino acid sequence selected from the group consisting of: SEQ ID NO: 2 and SEQ ID NO: 7 (S1scAb06); SEQ ID NO: 12 and SEQ ID NO: 17 (S1scAb11); SEQ ID NO: 22 and SEQ ID NO: 27 (S2H1); SEQ ID NO: 32 and SEQ ID NO: 37 (S2H2); SEQ ID NO: 42 and SEQ ID NO: 47 (S2H3); SEQ ID NO: 52 and SEQ ID NO: 57 (S2H4); SEQ ID NO: 62 and SEQ ID NO: 67 (S2H5); SEQ ID NO: 72 and SEQ ID NO: 77 (S2H6); and SEQ ID NO: 82 and SEQ ID NO: 87 (S2H7), respectively.

In one aspect, the present disclosure provides an antibody or antigen binding fragment thereof comprising (a) a light chain immunoglobulin variable domain sequence that is at least 80%, at least 85%, at least 90%, at least 95%, or at least 99% identical to the light chain immunoglobulin variable domain sequence of any one of SEQ ID NOs: 7, 17, 27, 37, 47, 57, 67, 77, or 87; and/or (b) a heavy chain immunoglobulin variable domain sequence (V_{H}) that is at least 80%, at least 85%, at least 90%, at least 95%, or at least 99% identical to the heavy chain immunoglobulin variable domain sequence present in any one of SEQ ID NOs: 2, 12, 22, 32, 42, 52, 62, 72 or 82.

Additionally or alternatively, in any of the embodiments disclosed herein, the antibody, or antigen binding fragment thereof, further comprises a Fc domain of an isotype selected from the group consisting of IgG1, IgG2, IgG3, IgG4, IgA1, IgA2, IgM, IgD, and IgE. Additionally or alternatively, in some embodiments, the antigen binding fragment is selected from the group consisting of Fab, F(ab′)₂, Fab′, scFᵥ, and Fᵥ. Additionally or alternatively, in some embodiments, the antibody is a monoclonal antibody, a chimeric antibody, a humanized antibody, or a bispecific antibody. Additionally or alternatively, in some embodiments, anti-leptin receptor antibody, or antigen binding fragment binds to the CRH2 domain of human leptin receptor. In some embodiments, the anti-leptin receptor antibody or antigen binding fragment of the present technology binds to a conformational epitope.

In one aspect, the present disclosure provides a method for treating a disorder associated with or caused by leptin deficiency or hypoleptinemia, leptin resistance, or leptin receptor mutations causing defective or impaired leptin signaling in a subject in need thereof, comprising: administering to the subject a therapeutically effective amount of an antibody or antigen binding fragment of the present technology.

In another aspect, the present technology provides a method for alleviating one or more symptoms of a disorder associated with or caused by leptin deficiency or hypoleptinemia, leptin resistance, or leptin receptor mutations causing defective or impaired leptin signaling in a subject in need thereof, comprising: administering to the subject a therapeutically effective amount of an antibody or antigen binding fragment disclosed herein. Examples of symptoms of such disorders include increased body weight, increased food intake, increased blood glucose levels, decreased insulin levels, decreased glucose tolerance, etc.

Additionally or alternatively, in some embodiments of the methods disclosed herein, the disorder associated with or caused by leptin receptor mutations causing defective or impaired leptin signaling is obesity.

In one aspect, the present disclosure provides a composition comprising the anti-leptin receptor antibody or antigen binding fragment of any of the embodiments disclosed herein.

In one aspect, the present disclosure provides a nucleic acid sequence encoding the antibody, or antigen binding fragment of any of the embodiments disclosed herein.

Additionally or alternatively, in some embodiments, the nucleic acid sequence is selected from the group consisting of SEQ ID NOs: 1, 6, 11, 16, 21, 26, 31, 36, 41, 46, 51, 56, 61, 66, 71, 76, 81, and 86.

In one aspect, the present disclosure provides a host cell or a vector expressing the nucleic acid.

In one aspect, the present disclosure provides a kit comprising the antibody, or antigen binding fragment of any one of the embodiments disclosed herein. Additionally or alternatively, in some embodiments, the antibody, or antigen binding fragment of the present technology is coupled to at least one detectable label selected from the group consisting of a radioactive label, a fluorescent label, and a chromogenic label. Additionally or alternatively, in some embodiments, the kit further comprises a secondary antibody that specifically binds to an antibody, or antigen binding fragment disclosed herein.

In one aspect, the present disclosure provides a method for detecting leptin receptor in a biological sample comprising contacting the biological sample with an antibody, or antigen binding fragment thereof disclosed herein, wherein the antibody or antigen binding fragment is conjugated to a detectable label; and detecting the levels of the detectable label in the biological sample.

### BRIEF DESCRIPTION OF THE DRAWINGS

FIG. 1A shows the effect of leptin or the anti-leptin receptor antibodies S1scAb06, S1scAb11, and S2H6 on the luciferase expression of cells harboring the SIS-inducible element (SIE)-luciferase vector. An isotype control antibody was used as a negative control.

FIG. 1B shows the effect of leptin or the anti-leptin receptor antibodies S2H1, S2H2, S2H3, S2H4, S2H5, S2H6, and S2H7 on the luciferase expression of cells harboring the SIS-inducible element (SIE)-luciferase vector. An isotype control antibody was used as a negative control.

FIG. 2A shows the effect of leptin or the anti-leptin receptor antibodies S1scAb06, S1scAb11, and S2H6 on the proliferation of the leptin-dependent Ba/F3-lepR reporter cells. An isotype control antibody was used as a negative control.

FIG. 2B shows the effect of leptin or the anti-leptin receptor antibodies S2H1, S2H2, S2H3, S2H4, S2H5, S2H6, and S2H7 on the proliferation of the leptin-dependent Ba/F3-lepR reporter cells. An isotype control antibody was used as a negative control.

FIG. 3A shows the effect of leptin or the anti-leptin receptor antibody S2H6 on the body weight of ob/ob mice. Six-week old female ob/ob mice were subcutaneously administered with vehicle (PBS, twice daily), leptin (0.5 mg/kg, twice daily) or S2H6 (5 mg/kg, once every other day) for two weeks (n=8). Body weights were monitored daily.

FIG. 3B shows the effect of leptin or the anti-leptin receptor antibody S2H6 on the food intake of ob/ob mice. Experiments were conducted as described in FIG. 3A. Food intake was monitored daily.

FIG. 3C shows the effect of leptin or the anti-leptin receptor antibody S2H6 on the blood glucose levels in ob/ob mice. Experiments were conducted as described in FIG. 3A. Blood Glucose levels were measured twice a week, and consecutive measurements are shown. ****: p<0.0001; ***: p=0.0001-0.001; ** p=0.001-0.01; * p=0.01-0.05.

FIG. 3D shows the effect of leptin or the anti-leptin receptor antibody S2H6 on the insulin levels in blood of ob/ob mice. Experiments were conducted as described in FIG. 3A. After two weeks trial, mice underwent fasting for 16 h, and blood insulin concentration was measured. ****: p<0.0001

FIG. 3E shows the effect of leptin or the anti-leptin receptor antibody S2H6 on the glucose tolerance by ob/ob mice. Experiments were conducted as described in FIG. 3A. After two weeks trial, mice underwent fasting for 16 h, and an intra-peritoneal glucose tolerance test (IPGTT) was performed to assess the body's ability to metabolize glucose.

FIG. 3F shows the effect of leptin or the anti-leptin receptor antibody S2H6 on the body fat present in ob/ob mice. Experiments were conducted as described in FIG. 3A. The mice were sacrificed after two weeks trial, the indicated adipose tissue was isolated and weighed. ****: p<0.0001; ***: p=0.0001-0.001; ** p=0.001-0.01; * p=0.01-0.05.

FIG. 4A shows the results of a competition assay between leptin and 51scAb06 antibody for binding to leptin receptor. Increasing concentrations of leptin (indicated on X-axis) and indicated fixed concentrations of S1scAb06 antibody were used in the assay. The results demonstrate that leptin can compete with the 51scAb06 antibody for binding to leptin receptor with an EC₅₀ of 6.55 nM.

FIG. 4B shows the results of a competition assay between leptin and S2H6 antibody for binding to leptin receptor. Increasing concentrations of leptin (indicated on X-axis) and indicated fixed concentrations of S2H6 antibody were used in the assay. The results demonstrate that leptin cannot compete with the S2H6 antibody for binding to leptin receptor.

FIGS. 5A-5D show the binding kinetics of leptin receptor agonists S1scAb06 (FIG. 5A), S1scAb11 (FIG. 5B), S2H6 (FIG. 5C), and leptin (FIG. 5D) to recombinant leptin receptor (extracellular domain) as determined using a BiacoreT200™ SPR (surface plasmon resonance) system. The line graphs depict change in the Resonance Units (RU, which reflects the change in analyte binding capacity of the surface) as a function of time, upon the addition of the indicated concentrations of the agonists.

FIG. 6 shows effect of leptin or the anti-leptin receptor antibodies on the activation of mutant leptin receptors as assayed using GFP expression by cells expressing the SIS-inducible element (SIE)-GFP reporter.

FIG. 7 shows that antibody S2H6 binds to the cytokine receptor homology domain. ELISA for binding of S2H6 with the following domains was performed: leptin receptor extracellular domain, N terminal domain (NTD), first cytokine receptor homology domain (CRH1), an immunoglobulin-like domain (IgD), a second cytokine receptor homology domain (CRH2) and fibronectin type III domains (FNIII).

FIG. 8A shows the nucleotide sequence of the V_{H} domain of the antibody S1scAb06 (SEQ ID NO: 1).

FIG. 8B shows the amino acid sequence of the V_{H} domain of the antibody S1scAb06 (SEQ ID NO: 2). The V_{H} CDR1 (SEQ ID NO: 3), V_{H} CDR2 (SEQ ID NO: 4), and V_{H} CDR3 (SEQ ID NO: 5) sequences are indicated by underlined boldface font.

FIG. 8C shows the nucleotide sequence of the V_{L} domain of the antibody S1scAb06 (SEQ ID NO: 6).

FIG. 8D shows the amino acid sequence of the V_{L} domain of the antibody S1scAb06 (SEQ ID NO: 7). The V_{L} CDR1 (SEQ ID NO: 8), V_{L} CDR2 (SEQ ID NO: 9), and V_{L} CDR3 (SEQ ID NO: 10) sequences are indicated by underlined boldface font.

FIG. 9A shows the nucleotide sequence of the V_{H} domain of the antibody S1scAb11 (SEQ ID NO: 11).

FIG. 9B shows the amino acid sequence of the V_{H} domain of the antibody S1scAb11 (SEQ ID NO: 12). The V_{H} CDR1 (SEQ ID NO: 13), V_{H} CDR2 (SEQ ID NO: 14), and V_{H} CDR3 (SEQ ID NO: 15) sequences are indicated by underlined boldface font.

FIG. 9C shows the nucleotide sequence of the V_{L} domain of the antibody S1scAb11 (SEQ ID NO: 16).

FIG. 9D shows the amino acid sequence of the V_{L} domain of the antibody S1scAb11 (SEQ ID NO: 17). The V_{L} CDR1 (SEQ ID NO: 18), V_{L} CDR2 (SEQ ID NO: 19), and V_{L} CDR3 (SEQ ID NO: 20) sequences are indicated by underlined boldface font.

FIG. 10A shows the nucleotide sequence of the V_{H} domain of the antibody S2H1 (SEQ ID NO: 21).

FIG. 10B shows the amino acid sequence of the V_{H} domain of the antibody S2H1 (SEQ ID NO: 22). The V_{H} CDR1 (SEQ ID NO: 23), V_{H} CDR2 (SEQ ID NO: 24), and V_{H} CDR3 (SEQ ID NO: 25) sequences are indicated by underlined boldface font.

FIG. 10C shows the nucleotide sequence of the V_{L} domain of the antibody S2H1 (SEQ ID NO: 26).

FIG. 10D shows the amino acid sequence of the V_{L} domain of the antibody S2H1 (SEQ ID NO: 27). The V_{L} CDR1 (SEQ ID NO: 28), V_{L} CDR2 (SEQ ID NO: 29), and V_{L} CDR3 (SEQ ID NO: 30) sequences are indicated by underlined boldface font.

FIG. 11A shows the nucleotide sequence of the V_{H} domain of the antibody S2H2 (SEQ ID NO: 31).

FIG. 11B shows the amino acid sequence of the V_{H} domain of the antibody S2H2 (SEQ ID NO: 32). The V_{H} CDR1 (SEQ ID NO: 33), V_{H} CDR2 (SEQ ID NO: 34), and V_{H} CDR3 (SEQ ID NO: 35) sequences are indicated by underlined boldface font.

FIG. 11C shows the nucleotide sequence of the V_{L} domain of the antibody S2H2 (SEQ ID NO: 36).

FIG. 11D shows the amino acid sequence of the V_{L} domain of the antibody S2H2 (SEQ ID NO: 37). The V_{L} CDR1 (SEQ ID NO: 38), V_{L} CDR2 (SEQ ID NO: 39), and V_{L} CDR3 (SEQ ID NO: 40) sequences are indicated by underlined boldface font.

FIG. 12A shows the nucleotide sequence of the V_{H} domain of the antibody S2H3 (SEQ ID NO: 41).

FIG. 12B shows the amino acid sequence of the V_{H} domain of the antibody S2H3 (SEQ ID NO: 42). The V_{H} CDR1 (SEQ ID NO: 43), V_{H} CDR2 (SEQ ID NO: 44), and V_{H} CDR3 (SEQ ID NO: 45) sequences are indicated by underlined boldface font.

FIG. 12C shows the nucleotide sequence of the V_{L} domain of the antibody S2H3 (SEQ ID NO: 46).

FIG. 12D shows the amino acid sequence of the V_{L} domain of the antibody S2H3 (SEQ ID NO: 47). The V_{L} CDR1 (SEQ ID NO: 48), V_{L} CDR2 (SEQ ID NO: 49), and V_{L} CDR3 (SEQ ID NO: 50) sequences are indicated by underlined boldface font.

FIG. 13A shows the nucleotide sequence of the V_{H} domain of the antibody S2H4 (SEQ ID NO: 51).

FIG. 13B shows the amino acid sequence of the V_{H} domain of the antibody S2H4 (SEQ ID NO: 52). The V_{H} CDR1 (SEQ ID NO: 53), V_{H} CDR2 (SEQ ID NO: 54), and V_{H} CDR33 (SEQ ID NO: 55) sequences are indicated by underlined boldface font.

FIG. 13C shows the nucleotide sequence of the V_{L} domain of the antibody S2H4 (SEQ ID NO: 56).

FIG. 13D shows the amino acid sequence of the V_{L} domain of the antibody S2H4 (SEQ ID NO: 57). The V_{L} CDR1 (SEQ ID NO: 58), V_{L} CDR2 (SEQ ID NO: 59), and V_{L} CDR3 (SEQ ID NO: 60) sequences are indicated by underlined boldface font.

FIG. 14A shows the nucleotide sequence of the V_{H} domain of the antibody S2H5 (SEQ ID NO: 61).

FIG. 14B shows the amino acid sequence of the V_{H} domain of the antibody S2H5 (SEQ ID NO: 62). The V_{H} CDR1 (SEQ ID NO: 63), V_{H} CDR2 (SEQ ID NO: 64), and V_{H} CDR3 (SEQ ID NO: 65) sequences are indicated by underlined boldface font.

FIG. 14C shows the nucleotide sequence of the V_{L} domain of the antibody S2H5 (SEQ ID NO: 66).

FIG. 14D shows the amino acid sequence of the V_{L} domain of the antibody S2H5 (SEQ ID NO: 67). The V_{L} CDR1 (SEQ ID NO: 68), V_{L} CDR2 (SEQ ID NO: 69), and V_{L} CDR3 (SEQ ID NO: 70) sequences are indicated by underlined boldface font.

FIG. 15A shows the nucleotide sequence of the V_{H} domain of the antibody S2H6 (SEQ ID NO: 71).

FIG. 15B shows the amino acid sequence of the V_{H} domain of the antibody S2H6 (SEQ ID NO: 72). The V_{H} CDR1 (SEQ ID NO: 73), V_{H} CDR2 (SEQ ID NO: 74), and V_{H} CDR3 (SEQ ID NO: 75) sequences are indicated by underlined boldface font.

FIG. 15C shows the nucleotide sequence of the V_{L} domain of the antibody S2H6 (SEQ ID NO: 76).

FIG. 15D shows the amino acid sequence of the V_{L} domain of the antibody S2H6 (SEQ ID NO: 77). The V_{L} CDR1 (SEQ ID NO: 78), V_{L} CDR2 (SEQ ID NO: 79), and V_{L} CDR3 (SEQ ID NO: 80) sequences are indicated by underlined boldface font.

FIG. 16A shows the nucleotide sequence of the V_{H} domain of the antibody S2H7 (SEQ ID NO: 81).

FIG. 16B shows the amino acid sequence of the V_{H} domain of the antibody S2H7 (SEQ ID NO: 82). The V_{H} CDR1 (SEQ ID NO: 83), V_{H} CDR2 (SEQ ID NO: 84), and V_{H} CDR3 (SEQ ID NO: 85) sequences are indicated by underlined boldface font.

FIG. 16C shows the nucleotide sequence of the V_{L} domain of the antibody S2H7 (SEQ ID NO: 86).

FIG. 16D shows the amino acid sequence of the V_{L} domain of the antibody S2H7 (SEQ ID NO: 87). The V_{L} CDR1 (SEQ ID NO: 88), V_{L} CDR2 (SEQ ID NO: 89), and V_{L} CDR3 (SEQ ID NO: 90) sequences are indicated by underlined boldface font.

### DETAILED DESCRIPTION

It is to be appreciated that certain aspects, modes, embodiments, variations and features of the present technology are described below in various levels of detail in order to provide a substantial understanding of the present technology.

### Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. In some embodiments, the anti-leptin receptor antibodies of the present technology are administered by an intracoronary route or an intra-arterial route. Administration includes self-administration and the administration by another.

As used herein, the term “amino acid” is used to refer to any organic molecule that contains at least one amino group and at least one carboxyl group. Typically, at least one amino group is at the α position relative to a carboxyl group. The term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refer to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W. H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds leptin receptor protein will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Anti-leptin receptor antibodies of the present technology” as used herein, refers to antibodies (including monoclonal antibodies, polyclonal antibodies, humanized antibodies, chimeric antibodies, recombinant antibodies, multispecific antibodies, bispecific antibodies, etc.) as well as antibody fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multispecific antibodies formed from antibody fragments.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fv (scFv)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide (e.g., a leptin receptor polypeptide). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFv-Fc, Fab, Fab′ and F(ab′)₂, but are not limited thereto.

Any of the above-noted antibody fragments are obtained using conventional techniques known to those of skill in the art, and the fragments are screened for binding specificity and neutralization activity in the same manner as are intact antibodies.

By “binding affinity” is meant the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs.

Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is an adipose tissue.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

An “isolated” or “purified” polypeptide or peptide is substantially free of cellular material or other contaminating polypeptides from the cell or tissue source from which the agent is derived, or substantially free from chemical precursors or other chemicals when chemically synthesized. For example, isolated anti-leptin receptor antibodies of the present technology would be free of materials that would interfere with diagnostic or therapeutic uses of the agent. Such interfering materials may include enzymes, hormones and other proteinaceous and nonproteinaceous solutes.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics.

Conformational and non-conformational epitopes are distinguished in that the binding to the former but not the latter is lost in the presence of denaturing solvents. In some embodiments, an “epitope” is the second cytokine receptor homology domain to which the anti-leptin receptor antibodies of the present technology specifically bind. In some embodiments, the epitope is a conformational epitope or a non-conformational epitope. To screen for anti-leptin receptor antibodies which bind to an epitope, a routine cross-blocking assay such as that described in Antibodies, A Laboratory Manual, Cold Spring Harbor Laboratory, Ed Harlow and David Lane (1988), can be performed. This assay can be used to determine if an anti-leptin receptor antibody binds the same site or epitope as an anti-leptin receptor antibody of the present technology. Alternatively, or additionally, epitope mapping can be performed by methods known in the art. For example, the antibody sequence can be mutagenized such as by alanine scanning, to identify contact residues. In a different method, peptides corresponding to different regions of leptin receptor protein can be used in competition assays with the test antibodies or with a test antibody and an antibody with a characterized or known epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the terms “Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Riechmann et al., Nature 332: 323-327 (1988); and Presta, Curr. Op. Struct. Biol. 2:593-596 (1992). See e.g., Ahmed & Cheung, FEBS Letters 588(2):288-297 (2014); Saxena & Wu, Frontiers in immunology 7: 580 (2016).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

As used herein, the terms “individual”, “patient”, or “subject” can be an individual organism, a vertebrate, a mammal, or a human. In some embodiments, the individual, patient or subject is a human.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴M, 10⁻⁵M, 10⁻⁶M, 10⁻⁷M, 10⁻⁸M, 10⁻⁹M, 10⁻¹⁰ M, 10⁻¹¹ M, or 10⁻¹²M. The term “specifically binds” may also refer to binding where a molecule (e.g., an antibody or antigen binding fragment thereof) binds to a particular polypeptide (e.g., a leptin receptor polypeptide), or an epitope on a particular polypeptide, without substantially binding to any other polypeptide, or polypeptide epitope.

As used herein, the terms “subject,” “individual,” or “patient” can be an individual organism, a vertebrate, a mammal, or a human.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. For example, a subject is successfully “treated” for obesity, leptin deficiency, leptin resistance, and/or hypoleptinemia, if, after receiving a therapeutic amount of the anti-leptin receptor antibodies of the present technology according to the methods described herein, the subject shows observable and/or measurable restoration of the function of the mutant leptin receptor.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-leptin receptor antibodies described herein are contemplated. For example, it may be desirable to improve the binding affinity and/or other biological properties of the antibody. Amino acid sequence variants of an anti-leptin receptor antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to obtain the antibody of interest, as long as the obtained antibody possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table below.

Amino Acid Substitutions

| Original | Exemplary | Conservative |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

One type of substitutional variant involves substituting one or more hypervariable region residues of a parent antibody. A convenient way for generating such substitutional variants involves affinity maturation using phage display. Specifically, several hypervariable region sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen. Such contact residues and neighboring residues are candidates for substitution according to the techniques elaborated herein. Once such variants are generated, the panel of variants is subjected to screening as described herein and antibodies with similar or superior properties in one or more relevant assays may be selected for further development.

Leptin, Leptin Receptor and the Disorders Associated with or Caused by Leptin Deficiency or Insufficiency

Leptin is a 16-kD Protein that Plays a Critical Role in the Regulation of Body weight by inhibiting food intake and stimulating energy expenditure. Defects in leptin production cause severe hereditary obesity in rodents and humans. In addition to its effects on body weight, leptin has a variety of other functions, including the regulation of hematopoiesis, angiogenesis, wound healing, and the immune and inflammatory response. The LEP gene is the human homolog of the gene (ob) mutant in the mouse “obese” phenotype. Leptin deficiency is characterized by severe early-onset obesity, hyperphagia, hypogonadotropic hypogonadism, and neuroendocrine/metabolic dysfunction. Ozata et al., J. Clin. Endocr. Metab. 84: 3686-3695 (1999).

Leptin acts through the leptin receptor (LEPR), a single-transmembrane-domain receptor of the cytokine receptor family, which is found in many tissues in several alternatively spliced forms. The leptin receptor gene, which is located at 1p31, encodes a single membrane spanning receptor of the class I cytokine receptor family. The disorders associated with or caused by leptin deficiency/insufficiency include hypoleptinemia, leptin resistance and the disorders caused by leptin receptor mutations leading to defective or impaired leptin signaling. For example, certain mutation in Leptin receptor (LEPR) results in severe, early onset obesity, diabetes. White and Tartaglia, Cytokine Growth Factor Rev 7:303-309 (1996); Chen et al. Cell 84:491-495 (1996); Morton and Schwartz, Physiol Rev 91:389-411 (2011); Bjorbaekand Kahn, Recent Prog Hormone Res 59:305-331 (2004); and Wauman and Tavernier, Front Biosci 17:2771-2793 (2012).

### Immunoglobulin-Related Compositions of the Present Technology

The present technology describes methods and compositions for the generation and use of anti-leptin receptor immunoglobulin-related compositions (e.g., anti-leptin receptor antibodies or antigen binding fragments thereof). The anti-leptin receptor antibodies of the present technology are agonists of leptin receptor; i.e., binding of anti-leptin receptor antibodies of the present technology to leptin receptor causes the activation of leptin receptor signaling. Accordingly, the anti-leptin receptor antibodies of the present technology are useful, e.g., for mimicking, substituting for, or supplementing the normal biological activity of leptin in a subject. The antibodies and antigen-binding fragments of the present technology are therefore useful in the therapeutic treatment of diseases and disorders associated with leptin resistance and leptin deficiency or dysfunction.

Accordingly, the anti-leptin receptor immunoglobulin-related compositions of the present disclosure may be useful in the diagnosis, or treatment of the disorders associated with defects in the leptin receptor, including obesity, diabetes, leptin deficiency, leptin resistance, and hypoleptinemia. Anti-leptin receptor immunoglobulin-related compositions within the scope of the present technology include, e.g., but are not limited to, monoclonal, chimeric, humanized, bispecific antibodies and diabodies that specifically bind the target polypeptide, a homolog, derivative or a fragment thereof. The present disclosure also provides antigen binding fragments of any of the anti-leptin receptor antibodies disclosed herein, wherein the antigen binding fragment is selected from the group consisting of Fab, F(ab)′2, Fab′, scFᵥ, and Fᵥ. The present technology discloses anti-leptin receptor antibodies formats that can activate leptin receptor mutants that are defective or impaired in leptin-binding or leptin-mediated signaling.

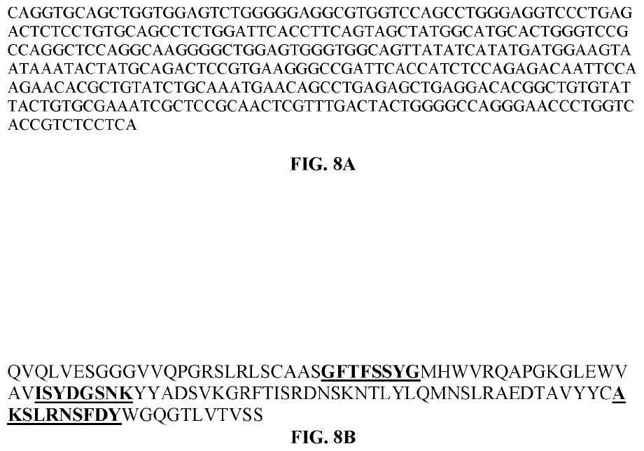

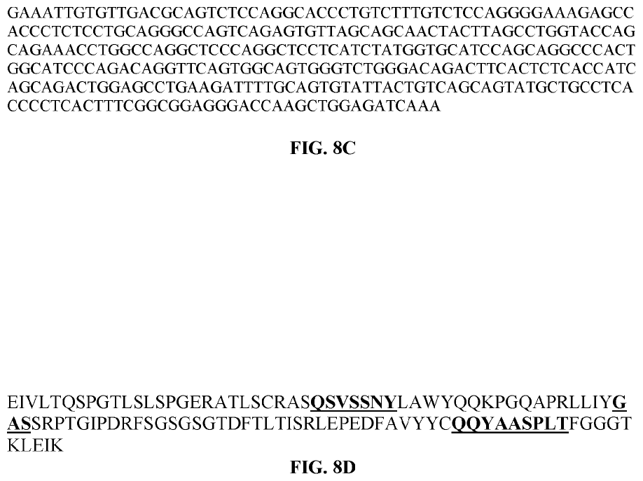

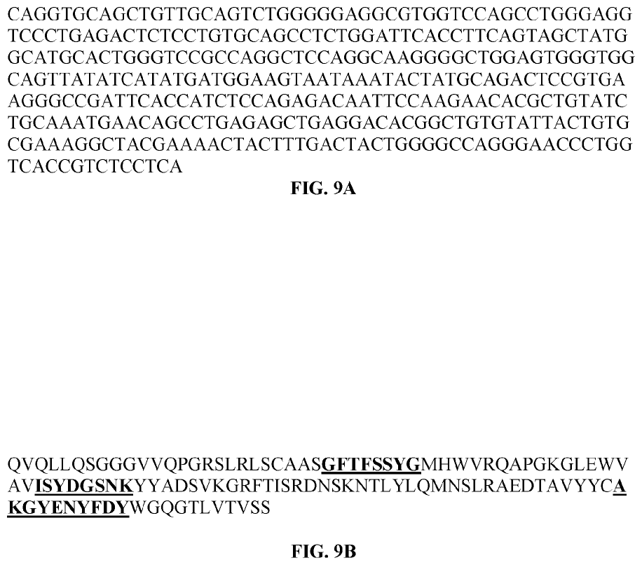

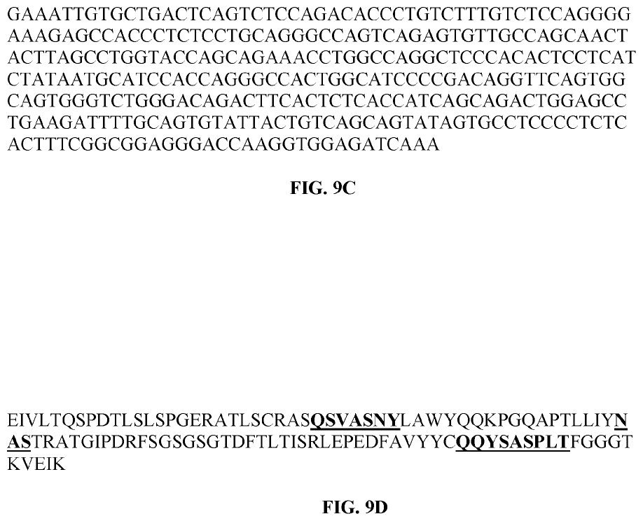

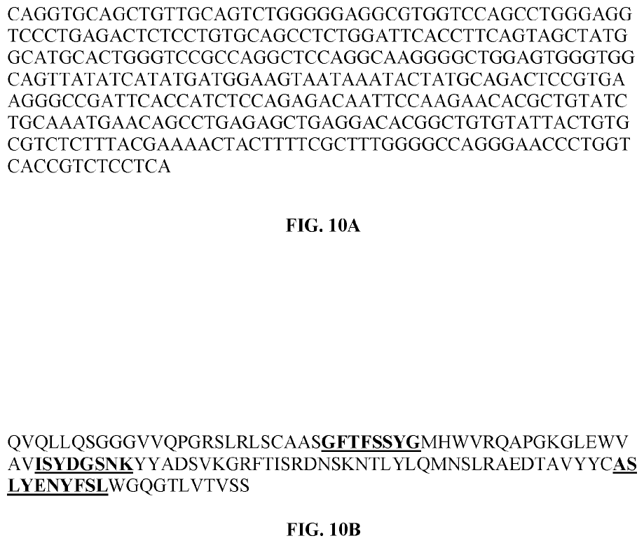

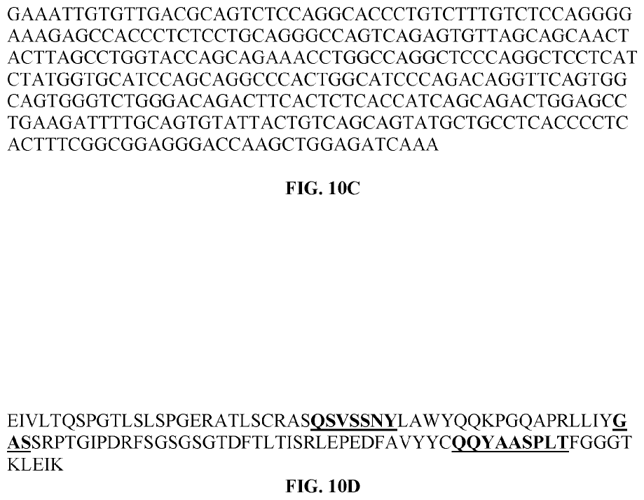

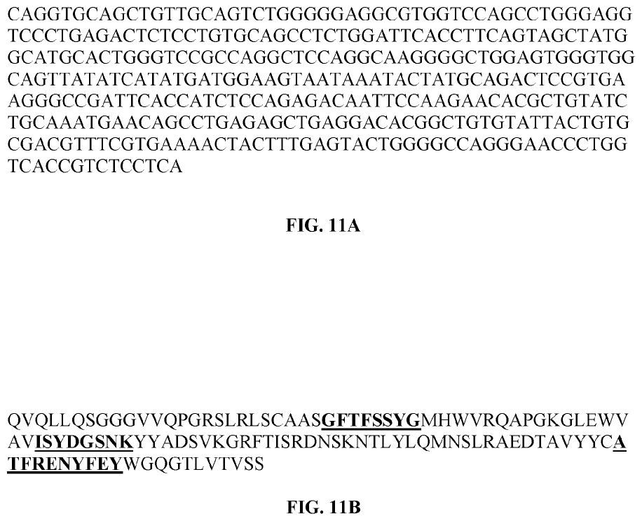

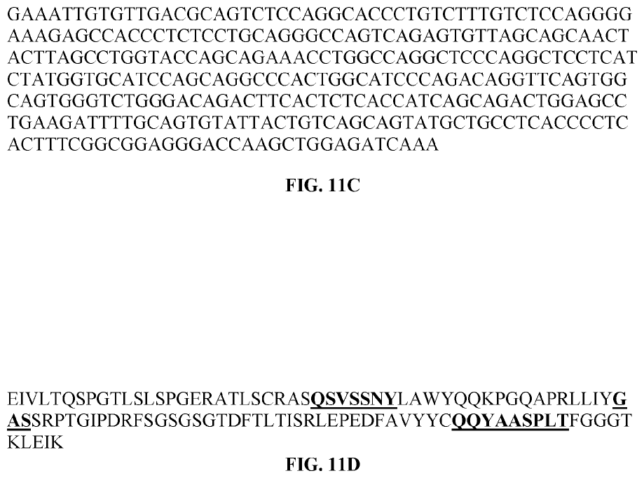

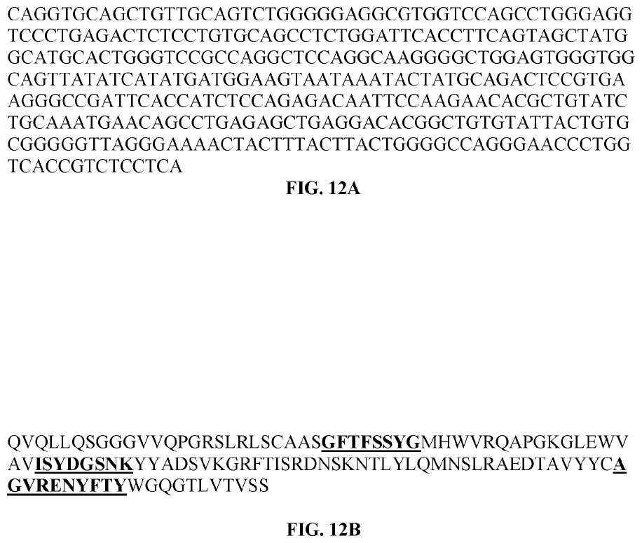

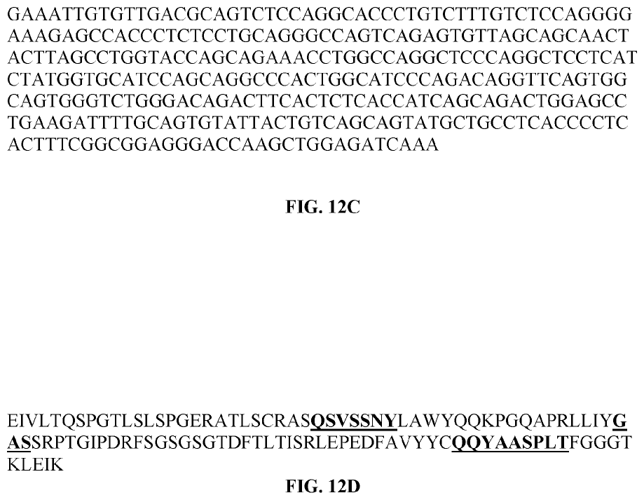

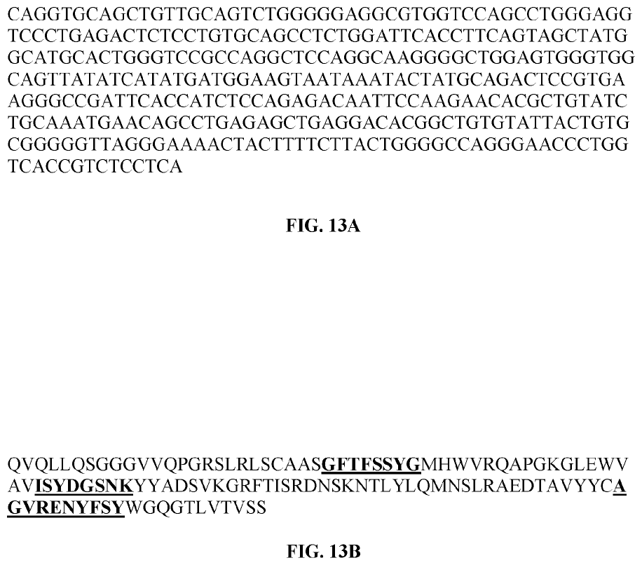

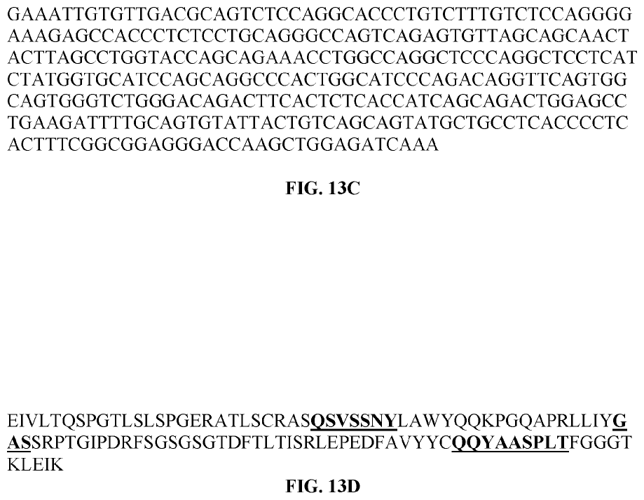

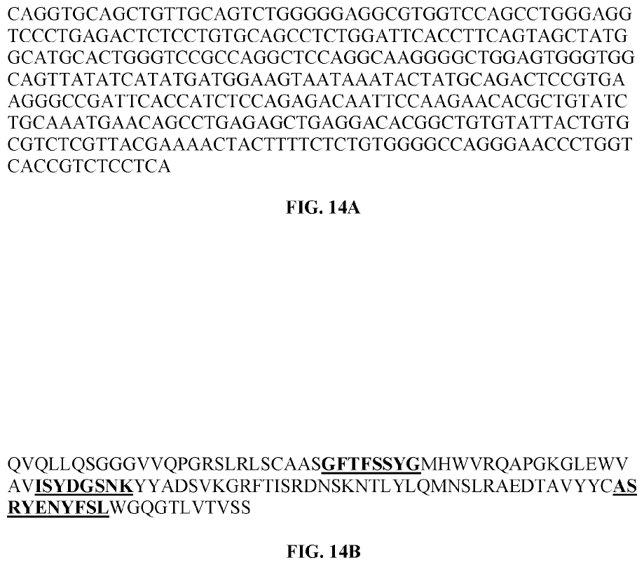

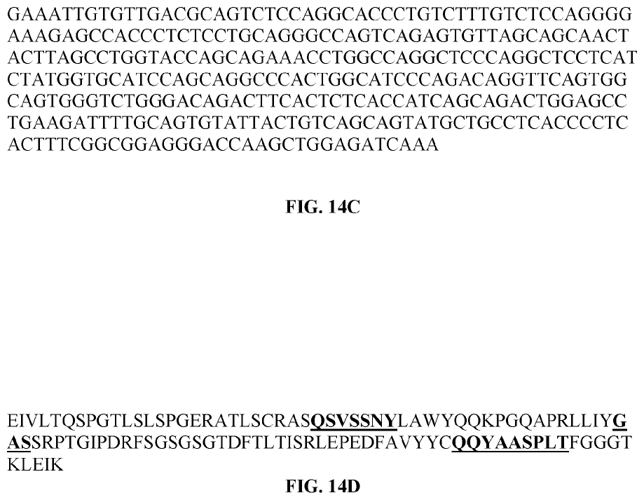

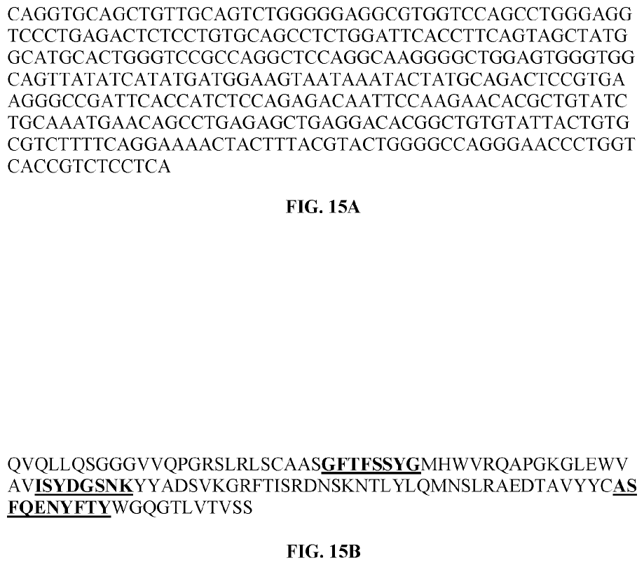

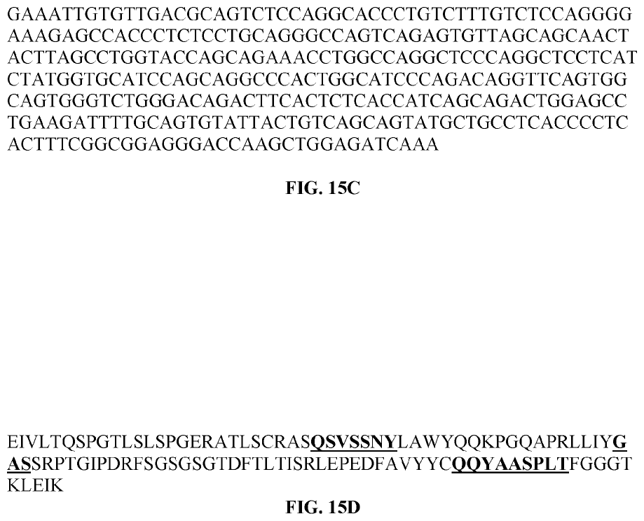

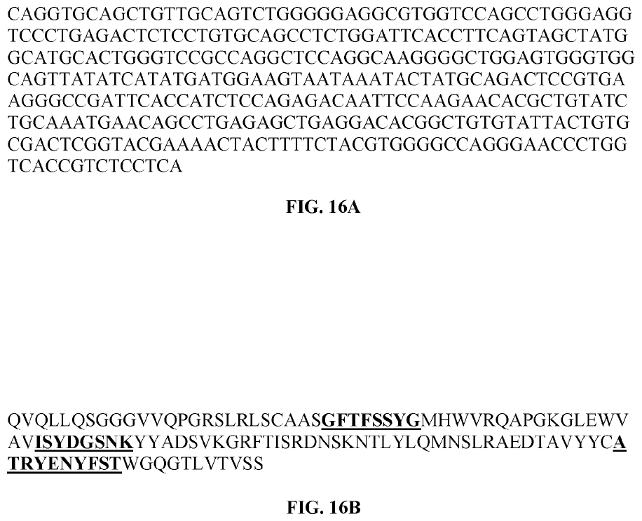

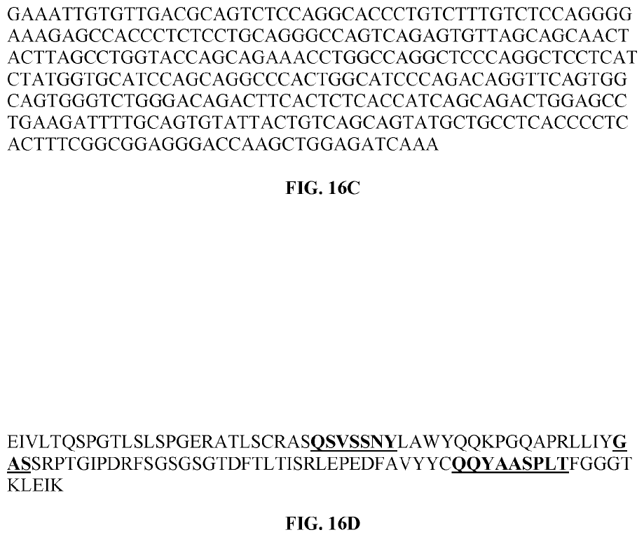

FIGS. 8-16 provides the nucleotide and amino acid sequences for V_{H} and V_{L} as well as the CDR sequences for the antibodies discloses herein (SEQ ID NOs: 1-90).

Present disclosure provides an anti-leptin receptor antibody, or antigen binding fragment thereof comprising a heavy chain immunoglobulin variable domain (V_{H}) and a light chain immunoglobulin variable domain (V_{L}), wherein the V_{H} comprises a V_{H}-CDR1 sequence selected from the group consisting of: SEQ ID NOs: 3, 13, 23, 33, 43, 53, 63, 73, and 83; a V_{H}-CDR2 sequence of selected from the group consisting of: SEQ ID NOs: 4, 14, 24, 34, 44, 54, 64, 74, and 84; and a V_{H}-CDR3 sequence selected from the group consisting of: SEQ ID NOs: 5, 15, 25, 35, 45, 55, 65, 75, and 85; and the V_{L} comprises an amino acid sequence selected from the group consisting of: a V_{L}-CDR1 sequence selected from the group consisting of: SEQ ID NOs: 8, 18, 28, 38, 48, 58, 68, 78, and 88; a V_{L}-CDR2 sequence of selected from the group consisting of: SEQ ID NOs: 9, 19, 29, 39, 49, 59, 69, 79, and 89; and a V_{H}-CDR3 sequence selected from the group consisting of: SEQ ID NOs: 10, 20, 30, 40, 50, 60, 70, 80, and 90. In some embodiments, the antibody further comprises a Fc domain of any isotype, e.g., but are not limited to, IgG (including IgG1, IgG2, IgG3, and IgG4), IgA (including IgA1 and IgA2), IgD, IgE, or IgM, and IgY. Non-limiting examples of constant region sequences include:

Human IgD constant region, Uniprot: P01880
(SEQ ID NO: 91)
APTKAPDVFPIISGCRHPKDNSPVVLACLITGYHPTSVTVTWYMGTQSQP
QRTFPEIQRRDSYYMTSSQLSTPLQQWRQGEYKCVVQHTASKSKKEIFRW
PESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDA
HLTWEVAGKVPTGGVEEGLLERHSNGSQSQHSRLTLPRSLWNAGTSVTCT
LNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQP
ATYTCVVSHEDSRTLLNASRSLEVSYVTDHGPMK
Human IgG1 constant region, Uniprot: P01857
(SEQ ID NO: 92)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human IgG2 constant region, Uniprot: P01859
(SEQ ID NO: 93)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVATHEALHNHYTQKSLSLSPGK
Human IgG3 constant region, Uniprot: P01860
(SEQ ID NO: 94)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK
Human IgM constant region, Uniprot: P01871
(SEQ ID NO: 95)
GSASAPTLFPLVSCENSPSDTSSVAVGCLAQDFLPDSITLSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGT
CY
Human IgG4 constant region, Uniprot: P01861
(SEQ ID NO: 96)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Human IgA1 constant region, Uniprot: P01876
(SEQ ID NO: 97)
ASPTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY
Human IgA2 constant region, Uniprot: P01877
(SEQ ID NO: 98)
ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRMAGKPTHVNVSVVMAEVDGTCY
Human Ig kappa constant region, Uniprot: P01834
(SEQ ID NO: 99)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

In some embodiments, the immunoglobulin-related compositions of the present technology comprise a heavy chain constant region that is at least 80%, at least 85%, at least 90%, at least 95%, at least 99%, or is 100% identical to SEQ ID NOS: 91-98. Additionally or alternatively, in some embodiments, the immunoglobulin-related compositions of the present technology comprise a light chain constant region that is at least 80%, at least 85%, at least 90%, at least 95%, at least 99%, or is 100% identical to SEQ ID NO: 99. In some embodiments, the immunoglobulin-related compositions of the present technology bind to the CRH2 domain of leptin receptor. In some embodiments, the epitope is a conformational epitope.

In another aspect, the present disclosure provides an isolated immunoglobulin-related composition (e.g., an antibody or antigen binding fragment thereof) comprising a V_{H} amino acid sequence comprising SEQ ID NO: 2, SEQ ID NO: 12, SEQ ID NO: 22, SEQ ID NO: 32, SEQ ID NO: 42, SEQ ID NO: 52, SEQ ID NO: 62, SEQ ID NO: 72, SEQ ID NO: 82, or a variant thereof having one or more conservative amino acid substitutions. Additionally or alternatively, in some embodiments, the immunoglobulin-related compositions of the present technology comprise a V_{L} amino acid sequence comprising SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, or a variant thereof having one or more conservative amino acid substitutions.

In some embodiments, the immunoglobulin-related compositions of the present technology comprise a V_{H} amino acid sequence and a V_{L} amino acid sequence selected from the group consisting of: SEQ ID NO: 2 and SEQ ID NO: 7 (S1scAb06); SEQ ID NO: 12 and SEQ ID NO: 17 (S1scAb11); SEQ ID NO: 22 and SEQ ID NO: 27 (S2H1); SEQ ID NO: 32 and SEQ ID NO: 37 (S2H2); SEQ ID NO: 42 and SEQ ID NO: 47 (S2H3); SEQ ID NO: 52 and SEQ ID NO: 57 (S2H4); SEQ ID NO: 62 and SEQ ID NO: 67 (S2H5); SEQ ID NO: 72 and SEQ ID NO: 77 (S2H6); SEQ ID NO: 82 and SEQ ID NO: 87 (S2H7); respectively.

In any of the above embodiments of the immunoglobulin-related compositions, the heavy chain (HC) and light chain (LC) immunoglobulin variable domain sequences form an antigen binding site that binds to the CRH2 domain of leptin receptor. In some embodiments, the epitope is a conformational epitope.

In some embodiments, the HC and LC immunoglobulin variable domain sequences are components of the same polypeptide chain. In other embodiments, the HC and LC immunoglobulin variable domain sequences are components of different polypeptide chains. In certain embodiments, the antibody is a full-length antibody.

In some embodiments, the immunoglobulin-related compositions of the present technology bind specifically to at least one leptin receptor polypeptide. In some embodiments, the immunoglobulin-related compositions of the present technology bind at least one leptin receptor polypeptide with a dissociation constant (K_{D}) of about 10⁻³M, 10⁻⁴ M, 10⁻⁵ M, 10⁻⁶ M, 10⁻⁷ M 10⁻⁸ M, 10⁻⁹ M, 10⁻¹⁰ M, 10⁻¹¹ M, or 10⁻¹² M. In certain embodiments, the immunoglobulin-related compositions are monoclonal antibodies, chimeric antibodies, humanized antibodies, or bispecific antibodies. In some embodiments, the antibodies comprise a human antibody framework region.

In certain embodiments, the immunoglobulin-related composition includes one or more of the following characteristics: (a) a light chain immunoglobulin variable domain sequence that is at least 80%, at least 85%, at least 90%, at least 95%, or at least 99% identical to the light chain immunoglobulin variable domain sequence present in any one of SEQ ID NOs: 7, 17, 27, 37, 47, 57, 67, 77, or 87; and/or (b) a heavy chain immunoglobulin variable domain sequence that is at least 80%, at least 85%, at least 90%, at least 95%, or at least 99% identical to the heavy chain immunoglobulin variable domain sequence present in any one of SEQ ID NOs: 2, 12, 22, 32, 42, 52, 62, 72 or 82. In another aspect, one or more amino acid residues in the immunoglobulin-related compositions provided herein are substituted with another amino acid. The substitution may be a “conservative substitution” as defined herein.

In some aspects, the anti-leptin receptor immunoglobulin-related compositions described herein contain structural modifications to facilitate rapid binding and cell uptake and/or slow release. In some aspects, the anti-leptin receptor immunoglobulin-related composition of the present technology (e.g., an antibody) may contain a deletion in the CH2 constant heavy chain region to facilitate rapid binding and cell uptake and/or slow release. In some aspects, a Fab fragment is used to facilitate rapid binding and cell uptake and/or slow release. In some aspects, a F(ab)′₂ fragment is used to facilitate rapid binding and cell uptake and/or slow release.

In one aspect, the present technology provides a nucleic acid sequence encoding any of the immunoglobulin-related compositions described herein. In some embodiments, the nucleic acid sequence is selected from the group consisting of SEQ ID NOs: 1, 6, 11, 16, 21, 26, 31, 36, 41, 46, 51, 56, 61, 66, 71, 76, 81, and 86.

In another aspect, the present technology provides a host cell or expression vector expressing any nucleic acid sequence encoding any of the immunoglobulin-related compositions described herein.

The immunoglobulin-related compositions of the present technology (e.g., an anti-leptin receptor antibody) can be monospecific, bispecific, trispecific or of greater multispecificity. Multispecific antibodies can be specific for different epitopes of one or more leptin receptor polypeptides or can be specific for both the leptin receptor polypeptide(s) as well as for heterologous compositions, such as a heterologous polypeptide or solid support material. See, e.g., WO 93/17715; WO 92/08802; WO 91/00360; WO 92/05793; Tutt et al., J. Immunol. 147: 60-69 (1991); U.S. Pat. Nos. 5,573,920, 4,474,893, 5,601,819, 4,714,681, 4,925,648; 6,106,835; Kostelny et al., J. Immunol. 148: 1547-1553 (1992). In some embodiments, the immunoglobulin-related compositions are chimeric. In certain embodiments, the immunoglobulin-related compositions are humanized.

The immunoglobulin-related compositions of the present technology can further be recombinantly fused to a heterologous polypeptide at the N- or C-terminus or chemically conjugated (including covalently and non-covalently conjugations) to polypeptides or other compositions. For example, the immunoglobulin-related compositions of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In any of the above embodiments of the immunoglobulin-related compositions of the present technology, the antibody or antigen binding fragment may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof. For a chemical bond or physical bond, a functional group on the immunoglobulin-related composition typically associates with a functional group on the agent. Alternatively, a functional group on the agent associates with a functional group on the immunoglobulin-related composition.

The functional groups on the agent and immunoglobulin-related composition can associate directly. For example, a functional group (e.g., a sulfhydryl group) on an agent can associate with a functional group (e.g., sulfhydryl group) on an immunoglobulin-related composition to form a disulfide. Alternatively, the functional groups can associate through a cross-linking agent (i.e., linker). Some examples of cross-linking agents are described below. The cross-linker can be attached to either the agent or the immunoglobulin-related composition. The number of agents or immunoglobulin-related compositions in a conjugate is also limited by the number of functional groups present on the other. For example, the maximum number of agents associated with a conjugate depends on the number of functional groups present on the immunoglobulin-related composition. Alternatively, the maximum number of immunoglobulin-related compositions associated with an agent depends on the number of functional groups present on the agent.

In yet another embodiment, the conjugate comprises one immunoglobulin-related composition associated to one agent. In one embodiment, a conjugate comprises at least one agent chemically bonded (e.g., conjugated) to at least one immunoglobulin-related composition. The agent can be chemically bonded to an immunoglobulin-related composition by any method known to those in the art. For example, a functional group on the agent may be directly attached to a functional group on the immunoglobulin-related composition. Some examples of suitable functional groups include, for example, amino, carboxyl, sulfhydryl, maleimide, isocyanate, isothiocyanate and hydroxyl.

The agent may also be chemically bonded to the immunoglobulin-related composition by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands.

Alternatively, the functional group on the agent and immunoglobulin-related composition can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidylsuberate), DMA (i.e., dimethyl adipimidate.2HC1), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the immunoglobulin-related composition. The web-site of Pierce Biotechnology, Inc. described above can also provide assistance to one skilled in the art in choosing suitable cross-linkers which can be cleaved by, for example, enzymes in the cell. Thus the agent can be separated from the immunoglobulin-related composition. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido)hexanoate), LC-SPDP (i.e., succinimidyl 6-(3-[2-pyridyldithio]-propionamido)hexanoate), Sulfo-LC-SPDP (i.e., sulfosuccinimidyl 6-(3-[2-pyridyldithio]-propionamido)hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one immunoglobulin-related composition. Any method known to those in the art can be employed to physically bond the agents with the immunoglobulin-related compositions. For example, the immunoglobulin-related compositions and agents can be mixed together by any method known to those in the art. The order of mixing is not important. For instance, agents can be physically mixed with immunoglobulin-related compositions by any method known to those in the art. For example, the immunoglobulin-related compositions and agents can be placed in a container and agitated, by for example, shaking the container, to mix the immunoglobulin-related compositions and agents.

The immunoglobulin-related compositions can be modified by any method known to those in the art. For instance, the immunoglobulin-related composition may be modified by means of cross-linking agents or functional groups, as described above.

### Formulations

By way of an example, anti-leptin receptor antibodies of the present technology is formulated in a simple delivery vehicle. However, anti-leptin receptor antibodies of the present technology may be lyophilized or incorporated in a gel, cream, biomaterial, sustained release delivery vehicle.

Anti-leptin receptor antibodies of the present technology are generally combined with a pharmaceutically acceptable carrier. The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids and amino acid copolymers. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Pharmaceutically acceptable salts can also be present in the pharmaceutical composition, e.g. mineral acid salts such as hydrochlorides, hydrobromides, phosphates, sulfates, and the like; and the salts of organic acids such as acetates, propionates, malonates, benzoates, and the like.

The anti-leptin receptor antibodies of the present technology may be provided in the form of a dressing. That is to say, anti-leptin receptor antibodies of the present technology is provided in the form of a liquid, semi-solid or solid composition for application directly to the skin surface, or the composition is applied to the surface of, or incorporated into, a solid skin contacting layer such as a dressing gauze or film. The dressing composition may be provided in the form of a fluid or a gel. The anti-leptin receptor antibodies of the present technology may be provided in combination with conventional pharmaceutical excipients for topical application to a wound. Suitable carriers include: Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g. creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilisers such as EDTA.

In some embodiments, the antibody or antigen binding fragment thereof is formulated as an ointment, salve, gel, or cream. In some embodiments, the antibody or antigen binding fragment thereof is formulated as an injectable.

### Modes of Administration and Effective Dosages

Any method known to those in the art for contacting a cell, organ or tissue with an immunoglobulin-related composition may be employed. Suitable methods include in vitro, ex vivo, or in vivo methods. In vivo methods typically include the administration of an anti-leptin receptor antibody of the present technology, such as those described above, to a mammal, suitably a human. When used in vivo for therapy, the anti-leptin receptor antibodies of the present technology are administered to the subject in effective amounts (i.e., amounts that have desired therapeutic effect). The dose and dosage regimen will depend upon the degree of the disease symptoms in the subject, the characteristics of the particular anti-leptin receptor antibodies of the present technology used, e.g., its therapeutic index, the subject, and the subject's history.

The effective amount may be determined during pre-clinical trials and clinical trials by methods familiar to physicians and clinicians. An effective amount of an immunoglobulin-related composition useful in the methods may be administered to a mammal in need thereof by any of a number of well-known methods for administering pharmaceutical compounds. The immunoglobulin-related composition may be administered systemically or locally.

The anti-leptin receptor antibodies of the present technology described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

In some embodiments, the anti-leptin receptor antibodies or antigen binding fragments of the present technology is administered by a parenteral route. In some embodiments, the antibody or antigen binding fragment thereof is administered by a topical route.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The immunoglobulin-related compositions described herein can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the immunoglobulin-related compositions of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of an immunoglobulin-related composition of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

An immunoglobulin-related composition of the present technology can be formulated in a carrier system. The carrier can be a colloidal system. The colloidal system can be a liposome, a phospholipid bilayer vehicle. In one embodiment, the therapeutic immunoglobulin-related compositionis encapsulated in a liposome while maintaining structural integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the anti-leptin receptor antibodies or antigen binding fragments of the present technology can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (SeeKozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the anti-leptin receptor antibodies or antigen binding fragments of the present technology are prepared with carriers that will protect the anti-leptin receptor antibodies or antigen binding fragments against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The anti-leptin receptor antibodies or antigen binding fragments of the present technology can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol 13(12):527-37 (1995). Mizguchi et al., Cancer Lett., 100:63-69 (1996), describes the use of fusogenic liposomes to deliver a protein to cells both in vivo and in vitro.

Dosage, toxicity and therapeutic efficacy of the anti-leptin receptor antibodies of the present technology can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the anti-leptin receptor antibodies or antigen binding fragments of the present technology exhibit high therapeutic indices. While anti-leptin receptor antibodies or antigen binding fragments of the present technology that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For any anti-leptin receptor antibodies or antigen binding fragments of the present technology used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the anti-leptin receptor antibodies or antigen binding fragments of the present technology, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of an immunoglobulin-related composition ranges from 0.001-10,000 micrograms per kg body weight. In one embodiment, anti-leptin receptor antibodies or antigen binding fragments of the present technology concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, and until the subject shows partial or complete amelioration of symptoms of disease. Thereafter, the patient can be administered a prophylactic regime.

In some embodiments, a therapeutically effective amount of an immunoglobulin-related composition of the present technology may be defined as a concentration of an immunoglobulin-related composition at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue. In some embodiments, the doses are administered by single daily or weekly administration, but may also include continuous administration (e.g., parenteral infusion or transdermal application). In some embodiments, the dosage of the immunoglobulin-related compositions of the present technology is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

For example, a therapeutically effective amount may partially or completely alleviate one or more symptoms of obesity, leptin deficiency, leptin resistance, and/or hypoleptinemia, including increased body weight, increased food intake, increased blood glucose levels, decreased insulin levels, decreased glucose tolerance, etc.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### Use of the Anti-Leptin Receptor Antibodies of the Present Technology

General. The anti-leptin receptor antibodies of the present technology are useful in methods known in the art relating to the localization and/or quantitation of leptin receptor protein or a mutant thereof (e.g., for use in measuring levels of the leptin receptor within appropriate physiological samples, for use in diagnostic methods, for use in imaging the polypeptide, and the like). The anti-leptin receptor antibodies of the present technology are useful to isolate a leptin receptor by standard techniques, such as affinity chromatography or immunoprecipitation. The anti-leptin receptor antibodies of the present technology can facilitate the purification of natural immunoreactive leptin receptor from biological samples, e.g., mammalian sera or cells as well as recombinantly-produced immunoreactive leptin receptor expressed in a host system. Moreover, anti-leptin receptor antibodies of the present technology can be used to detect an immunoreactive leptin receptor (e.g., in plasma, a cellular lysate or cell supernatant) in order to evaluate the abundance and pattern of expression of the immunoreactive polypeptide. The anti-leptin receptor antibodies of the present technology can be used diagnostically to monitor immunoreactive leptin receptor levels in tissue as part of a clinical testing procedure, e.g., to determine the efficacy of a given treatment regimen. As noted above, the detection can be facilitated by coupling (i.e., physically linking) the anti-leptin receptor antibodies of the present technology to a detectable substance.

Detection of leptin receptor. An exemplary method for detecting the presence or absence of an immunoreactive leptin receptor in a biological sample involves obtaining a biological sample from a test subject and contacting the biological sample with the anti-leptin receptor antibodies of the present technology capable of detecting an immunoreactive leptin receptor such that the presence of an immunoreactive leptin receptor is detected in the biological sample. Detection may be accomplished by means of a detectable label attached to the antibody.

The term “labeled” with regard to the anti-leptin receptor antibodies of the present technology is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In some embodiments, the anti-leptin receptor antibodies of the present technology disclosed herein are conjugated to one or more detectable labels. For such uses, the anti-leptin receptor antibodies of the present technology may be detectably labeled by covalent or non-covalent attachment of a chromogenic, enzymatic, radioisotopic, isotopic, fluorescent, toxic, chemiluminescent, nuclear magnetic resonance contrast agent or other label.

Examples of suitable chromogenic labels include diaminobenzidine and 4-hydroxyazo-benzene-2-carboxylic acid. Examples of suitable enzyme labels include malate dehydrogenase, staphylococcal nuclease, Δ-5-steroid isomerase, yeast-alcohol dehydrogenase, α-glycerol phosphate dehydrogenase, triose phosphate isomerase, peroxidase, alkaline phosphatase, asparaginase, glucose oxidase, β-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is an exemplary isotope where in vivo imaging is used since its avoids the problem of dehalogenation of the ¹²⁵I or ¹³¹I-labeled leptin receptor-protein binding antibodies by the liver. In addition, this isotope has a more favorable gamma emission energy for imaging (Perkins et al, Eur. J. Nucl. Med. 70:296-301 (1985); Carasquillo et al., J. Nucl. Med. 25:281-287 (1987)). For example, ¹¹¹In coupled to monoclonal antibodies with 1-(P-isothiocyanatobenzyl)-DPTA exhibits little uptake in non-tumorous tissues, particularly the liver, and enhances specificity of tumor localization (Esteban et al., J. Nucl. Med. 28:861-870 (1987)). Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label. Examples of suitable toxin labels include diphtheria toxin, ricin, and cholera toxin.

Examples of chemiluminescent labels include a luminol label, an isoluminol label, an aromatic acridinium ester label, an imidazole label, an acridinium salt label, an oxalate ester label, a luciferin label, a luciferase label, and an aequorin label. Examples of nuclear magnetic resonance contrasting agents include heavy metal nuclei such as Gd, Mn, and iron.

The detection method of the present technology can be used to detect an immunoreactive leptin receptor in a biological sample in vitro as well as in vivo. In vitro techniques for detection of an immunoreactive leptin receptor include enzyme linked immunosorbent assays (ELISAs), Western blots, immunoprecipitations, radioimmunoassay, and immunofluorescence. Furthermore, in vivo techniques for detection of an immunoreactive leptin receptor include introducing into a subject a labeled anti-leptin receptor antibody. For example, the anti-leptin receptor antibodies of the present technology can be labeled with a radioactive marker whose presence and location in a subject can be detected by standard imaging techniques. In one embodiment, the biological sample contains leptin receptor molecules from the test subject.

Immunoassay and Imaging. The anti-leptin receptor antibodies of the present technology can be used to assay immunoreactive leptin receptor levels in a biological sample (e.g., human plasma) using antibody-based techniques. For example, protein expression in tissues can be studied with classical immunohistological methods. Jalkanen, M. et al., J. Cell. Biol. 101: 976-985, 1985; Jalkanen, M. et al., J. Cell. Biol. 105: 3087-3096, 1987. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (³H), indium (¹¹²In), and technetium (⁹⁹mTc), and fluorescent labels, such as fluorescein, rhodamine, and green fluorescent protein (GFP), as well as biotin.

In addition to assaying immunoreactive leptin receptor levels in a biological sample, the anti-leptin receptor antibodies of the present technology may be used for in vivo imaging of leptin receptor. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the anti-leptin receptor antibodies of the present technology by labeling of nutrients for the relevant scFv clone.

An anti-leptin receptor antibody of the present technology which has been labeled with an appropriate detectable imaging moiety, such as a radioisotope (e.g., ¹³¹I, ¹¹²In, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled anti-leptin receptor antibody will then accumulate at the location of cells which contain the specific target polypeptide. For example, labeled anti-leptin receptor antibodies of the present technology will accumulate within the subject in cells and tissues in which the leptin receptor has localized.

Thus, the present technology provides a diagnostic method of a medical condition, which involves: (a) assaying the expression of immunoreactive leptin receptor by measuring binding of the anti-leptin receptor antibodies of the present technology in cells or body fluid of an individual; (b) comparing the amount of immunoreactive leptin receptor present in the sample with a standard reference, wherein an increase or decrease in immunoreactive leptin receptor levels compared to the standard is indicative of a medical condition.

Affinity Purification. The anti-leptin receptor antibodies of the present technology may be used to purify immunoreactive leptin receptor from a sample. In some embodiments, the antibodies are immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10 (1986); Jacoby et al., Meth. Enzym. 34 Academic Press, N.Y. (1974)).

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating or rocking the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

An antibody or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed.

For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. An antibody or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

### A. Diagnostic Uses of the Anti-Leptin Receptor Antibodies of the Present Technology

General. The anti-leptin receptor antibodies of the present technology are useful in diagnostic methods. As such, the present technology provides methods using the antibodies in the diagnosis of leptin receptor activity in a subject. The anti-leptin receptor antibodies of the present technology may be selected such that they have any level of epitope binding specificity and very high binding affinity to a leptin receptor. In general, the higher the binding affinity of an antibody the more stringent wash conditions can be performed in an immunoassay to remove nonspecifically bound material without removing target polypeptide.

Accordingly, the anti-leptin receptor antibodies of the present technology useful in diagnostic assays usually have binding affinities of about 10⁸M⁻¹, 10⁹M⁻¹, 10¹⁰ M⁻¹, 10¹¹ M⁻¹ or 10¹²M⁻¹. Further, it is desirable that the anti-leptin receptor antibodies of the present technology used as diagnostic reagents have a sufficient kinetic on-rate to reach equilibrium under standard conditions in at least 12 h, at least five (5) h, or at least one (1) hour.

The anti-leptin receptor antibodies of the present technology can be used to detect an immunoreactive leptin receptor in a variety of standard assay formats. Such formats include immunoprecipitation, Western blotting, ELISA, radioimmunoassay, and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue or body fluid of a subject. In certain embodiments, the subject is at an early stage of cancer. In one embodiment, the early stage of cancer is determined by the level or expression pattern of leptin receptor in a sample obtained from the subject. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. See U.S. Pat. Nos. 4,376,110, 4,486,530, 5,914,241, and 5,965,375. Such assays use one antibody, e.g., the anti-leptin receptor antibody or a population of the anti-leptin receptor antibodies immobilized to a solid phase, and another the anti-leptin receptor antibody or a population of anti-leptin receptor antibodies in solution. Typically, the solution anti-leptin receptor antibody or population of the anti-leptin receptor antibodies is labeled. If an antibody population is used, the population can contain antibodies binding to different epitope specificities within the target polypeptide. Accordingly, the same population can be used for both solid phase and solution antibody. If the anti-leptin receptor antibodies of the present technology are used, first and second leptin receptor monoclonal antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the leptin receptor with the anti-leptin antibody, a sample is incubated for a period that usually varies from about 10 min to about 24 hr and is usually about 1 hr. A wash step is then performed to remove components of the sample not specifically bound to the anti-leptin receptor antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody.

Usually for a given pair of antibodies or populations of antibodies and given reaction conditions, a calibration curve is prepared from samples containing known concentrations of target antigen. Concentrations of the immunoreactive leptin receptor in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the leptin receptor in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, the anti-leptin receptor antibodies of the present technology can be joined to a linker molecule, such as biotin for attachment to a surface bound linker, such as avidin.

In some embodiments, the present disclosure provides the anti-leptin receptor antibodies of the present technology conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells containing the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). U.S. Pat. No. 6,331,175 describes MRI technique and the preparation of antibodies conjugated to a MRI enhancing agent and is incorporated in its entirety by reference. In some embodiments, the diagnostic agents are selected from the group consisting of radioisotopes, enhancing agents for use in magnetic resonance imaging, and fluorescent compounds. In order to load an antibody component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose. Chelates may be coupled to the antibodies of the present technology using standard chemistries. The chelate is normally linked to the antibody by a group which enables formation of a bond to the molecule with minimal loss of immunoreactivity and minimal aggregation and/or internal cross-linking. Other methods and reagents for conjugating chelates to antibodies are disclosed in U.S. Pat. No. 4,824,659. Particularly useful metal-chelate combinations include 2-benzyl-DTPA and its monomethyl and cyclohexyl analogs, used with diagnostic isotopes for radio-imaging. The same chelates, when complexed with non-radioactive metals, such as manganese, iron and gadolinium are useful for MRI, when used along with the leptin receptor antibodies of the present technology.

### B. Therapeutic Use of the Anti-Leptin Receptor Antibodies of the Present Technology

General. The anti-leptin receptor antibodies of the present technology are agonists of leptin receptor; i.e., binding of anti-leptin receptor antibodies of the present technology to leptin receptor causes the activation of leptin receptor signaling. Accordingly, the anti-leptin receptor antibodies of the present technology are useful, e.g., for mimicking, substituting for, or supplementing the normal biological activity of leptin in a subject. The antibodies and antigen-binding fragments of the present technology are therefore useful in the therapeutic treatment of diseases and disorders associated with leptin resistance and leptin deficiency or dysfunction.

The present technology includes antibodies and antigen-binding fragments thereof that bind human leptin receptor and activate leptin receptor signaling. In the context of the present technology, “activation of leptin receptor signaling” means the stimulation of an intracellular effect that normally results from the interaction of leptin with leptin receptor in cells that express leptin receptor. In certain embodiments, “activation of leptin receptor signaling” means the transcriptional activation of STAT3, which can be detected using any method that can measure or identify, directly or indirectly, STAT3 activity, e.g., using a labeled version of STAT3 expressed in a reporter cell line. For example, the present technology includes antibodies and antigen-binding fragments thereof that activate leptin receptor signaling in a cell-based reporter assay, e.g., using a cell based assay format as defined in Example 7 herein, or a substantially similar assay. The activation of leptin receptor signaling may be assayed using a reporter cell line that for sensing phosphorylated STAT3, or induction of gene expression via the SIE element (sis-inducible element) as discussed in the Examples, particularly in Examples 1-3.

In some aspects, the anti-leptin receptor antibodies of the present technology are useful in methods disclosed herein provide therapies for the prevention, amelioration or treatment of a condition associated with decreased activity of leptin receptors.

In some embodiments, the condition associated with decreased activity of leptin receptors is obesity, leptin deficiency, leptin resistance, and/or hypoleptinemia. In some embodiments, the condition associated with decreased activity of leptin receptors is a genetic disorder is associated with a mutation in the leptin receptor. In some embodiments, the genetic disorder is obesity. Non-limiting examples of such leptin receptor mutations include Q223R, P316T, L372A, A409E, L505/506S, R612H, W664R, and H684P.

In one aspect, the present technology provides a method for treating a disorder associated with or caused by leptin deficiency or hypoleptinemia, leptin resistance, or leptin receptor mutations causing defective or impaired leptin signaling in a subject in need thereof, comprising: administering to the subject a therapeutically effective amount of an antibody or antigen binding fragment disclosed herein. Examples of such disorders include obesity.

In one aspect, the present technology provides a method for alleviating one or more symptoms of a disorder associated with or caused by leptin deficiency or hypoleptinemia, leptin resistance, or leptin receptor mutations causing defective or impaired leptin signaling in a subject in need thereof, comprising: administering to the subject a therapeutically effective amount of an antibody or antigen binding fragment disclosed herein. Examples of symptoms of such disorders include increased body weight, increased food intake, increased blood glucose levels, decreased insulin levels, decreased glucose tolerance, etc.

In some embodiments, anti-leptin receptor antibodies of the present technology are leptin receptor agonists. Thus, for example, one or more of the anti-leptin receptor antibodies of the present technology may be: (1) co-formulated and administered or delivered alone or simultaneously in a combined formulation with other active agents or the anti-leptin receptor antibodies of the present technology; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used. Administering such combinations of the anti-leptin receptor antibodies of the present technology and other active agents can result in synergistic biological effects when administered in a therapeutically effective amount to a subject suffering from a disorder associated with or caused by leptin deficiency or hypoleptinemia, leptin resistance, or leptin receptor mutations causing defective or impaired leptin signaling. An advantage of such an approach is that lower doses of the anti-leptin receptor antibodies of the present technology and/or other active agents may be needed to prevent, ameliorate or treat a subject suffering from, or predisposed to, obesity, leptin deficiency, leptin resistance, and/or hypoleptinemia. Further, potential side-effects of treatment may be avoided by use of lower dosages of the anti-leptin receptor antibodies of the present technology and/or other active agents.

The anti-leptin receptor antibodies of the present technology may be co-formulated with and/or administered in combination with one or more additional therapeutically active component(s), such as. e.g., pharmaceutical products prescribed for the treatment of obesity, hypercholesterolemia, hyperlipidemia, type 2 diabetes, type 1 diabetes, appetite control, infertility, etc. Examples of such additional therapeutically active components include, e.g., recombinant human leptin (e.g., metreleptin [MYALEP1]), PCSK9 inhibitors (e.g., anti-PCSK9 antibodies [alirocumab, evolocumab, bococizumab, lodelcizumab, ralpancizumab, etc.]), statins (atorvastatin, rosuvastatin, cerivastatin, pitavastatin, fluvastatin, simvastatin, lovastatin, pravastatin, etc.), ezetimibe, insulin, insulin variants, insulin secretagogues, metformin, sulfonylureas, sodium glucose cotransporter 2 (SGLT2) Inhibitors (e.g., dapaglifozin, canaglifozin, empagliflozin, etc.), GLP-1 agonists/analogues (e.g., extendin-4, exenatide, liraglutide, lixisenatide, albiglutide, dulaglutide, etc.), glucagon (GCG) inhibitors (e.g., anti-GCG antibodies), glucagon receptor (GCGR) inhibitors (e.g., anti-GCGR antibodies, small molecule GCGR antagonists, GCGR-specific antisense oligonucleotides, anti-GCGR aptamers [e.g., Spiegelmers], etc.), angiopoietin-like protein (ANGPTL) inhibitors (e.g., anti-ANGPTL3 antibodies, anti-ANGPTL4 antibodies, anti-ANGPTL8 antibodies, etc.), Phentermine, Orlistat, Topiramate, Bupropion, Topiramate/Phentermine, Bupropion/Naltrexone, Bupropion/Zonisamide, Pramlintide/Metrelepin, Lorcaserin, Cetilistat, Tesofensine, Velneperit, etc.

### Determination of the Biological Effect of the Anti-Leptin Receptor Antibodies of the Present Technology.

In various embodiments, suitable in vitro or in vivo assays are performed to determine the effect of a specific therapeutic based on an anti-leptin receptor antibody of the present technology and whether its administration is indicated for treatment. In various embodiments, in vitro assays can be performed with representative cell lines. In various embodiments, in vivo assays can be performed with representative animal models, such as mice harboring a mutant leptin receptor (e.g., having one or more of L372A, A409E, L505/506S mutations). These experiments may be used to determine if a given anti-leptin receptor antibody of the present technology exerts the desired effect in promoting the signal transduction activity of mutantleptin receptors, or restoration of the function of the mutant leptin receptor.

Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

In some embodiments, leptin receptor activity is determined by assays well known in the art. Peng et al. (2015), Chemistry & Biology 22: 1-10 (2015): and Bhaskar et al., Obesity 24: 1687-1694 (2016). In some embodiments, leptin receptor activity is determined by assays that measure biological activity in animal models harboring leptin receptor mutations such as L372A, A409E, or L505/506S. In some embodiments, leptin receptor activity is determined using assays that measure the rescue of mutant phenotype of the animal models.

### C. Kits

The present technology provides kits for the detection and/or treatment of a mutant leptin receptor associated disease, comprising at least one immunoglobulin-related composition of the present technology (e.g., any antibody or antigen binding fragment described herein), or a functional variant (e.g., substitutional variant) thereof. Optionally, the above described components of the kits of the present technology are packed in suitable containers and labeled for diagnosis and/or treatment of a mutant leptin receptor associated disease. The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kits are useful for detecting the presence of an immunoreactive leptin receptor in a biological sample, e.g., any body fluid including, but not limited to, e.g., serum, plasma, lymph, cystic fluid, urine, stool, cerebrospinal fluid, ascitic fluid or blood and including biopsy samples of body tissue. For example, the kit can comprise: one or more humanized, chimeric, or bispecific anti-leptin receptor antibodies of the present technology (or antigen binding fragments thereof) capable of binding a leptin receptor in a biological sample; means for determining the amount of the leptin receptor in the sample; and means for comparing the amount of the immunoreactive leptin receptor in the sample with a standard. One or more of the anti-leptin receptor antibodies may be labeled. The kit components, (e.g., reagents) can be packaged in a suitable container. The kit can further comprise instructions for using the kit to detect the immunoreactive leptin receptor.

For antibody-based kits, the kit can comprise, e.g., 1) a first antibody, e.g. a humanized, or chimeric leptin receptor or antibody of the present technology (or an antigen binding fragment thereof), attached to a solid support, which binds to a leptin receptor; and, optionally; 2) a second, different antibody which binds to either the leptin receptor or to the first antibody, and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for detection of a leptin receptor in vitro or in vivo, or for treatment of a mutant leptin receptor-associated disease in a subject in need thereof. In certain embodiments, the use of the reagents can be according to the methods of the present technology.

### EXAMPLES

The present technology is further illustrated by the following examples, which should not be construed as limiting in any way. For each of the examples below, any immunologic binding agent, such as IgG, IgM, IgA, IgD, IgE, and genetically modified IgG, and fragments thereof described herein could be used. By way of example, but not by limitation, the scFv-Fc antibodies used in the examples below could be S1scAb06, S1scAb11, S2H1, S2H2, S2H3, S2H4, S2H5, S2H6, S2H7, etc.

### Example 1: Antibody Generation

For antibody selection, phage display and phenotypic selection were combined with a reporter cell line that for sensing phosphorylated STATS. Briefly, a single-chain combinatorial antibody library was enriched after two round panning with recombinant leptin receptor extracellular domain to get a sub-library of smaller but more specific clones. After two rounds of phage panning, —10⁶ colonies were selected and phagemids were extracted. The antibody coding sequences were digested using the restriction enzymes SfiI and cloned into a lentivirus vector, which is a member of the tethered system, for allowing mammalian cell surface display. For the selection agonist antibody from sub-library a beta-lactamase LepR reporter cell line was used, as this cell line provided a very good signal to noise ratio fora readout for sensing phosphorylated STAT3. The beta-lactamase LepR reporter cell line was infected with lentiviral libraries at MOI=2. After 8 hr of inoculation, the media were replaced and the cells were cultured for another 40 hr. Reporter cells were collected and incubated with LiveBLAzer™-FRET substrate CCF4-AM (Invitrogen) for 2 hr in dark, washed with FACS buffer and subjected to single cell sorting. β-lactamase positive single cell clones were allowed to reach confluence, and the antibody genes from each colony were amplified by PCR based on sequences from the lentiviral vector. The closes were sequenced. Sequence analysis revealed two promising closes, named S1scAb06 and S1scAb11, which showed maximal phosphorylated STAT3, which is indicative of LepR activation. Here, “S1” in antibody names refers to the first round of selection of antibody agonistic to the human leptin receptor.

### Example 2: Directed Evolution of Antibodies

A directed evolution approach was used by employing yeast display and flow cytometry for the selection higher affinity antibodies. In brief, a stop codon was introduced in the S1scAb06 nucleic acid at the location corresponding to the V_{H}CDR3 to generate a template antibody sequence for mutation library construction. Codons for four amino acids in V_{H}CDR3 were substituted with degenerate codons NNK (where, N=A/C/G/T & K=G/T) to construct a mutant antibody library with ˜10⁷ different protein sequences. Yeast cells carrying scFv antibody library were cultured in SD/Trp⁻ media to logarithmic phase at 30° C. with shaking. Yeast cells were then grown SGR-CAA medium for 24 h at 20° C. with shaking to induce yeast display. A recombinant leptin receptor extracellular domain fused to His tag was purified and labeled with biotin using the EZ-LINK NHS-PEG4-BIOTIN kit. The biotin-labelled recombinant leptin receptor extracellular domain protein was used as an antigen to bind the yeast antibody library and higher affinity hits were selected with 3 rounds of flow cytometry. Antibody sequences in yeast display plasmids from final round were extracted and sequenced. Seven hits were obtained from yeast were named S2H1 through S2H7. Here, “S2” in antibody names refers to the second round of selection of antibody agonistic to the human leptin receptor.

The Table below and FIGS. 8-16 provides the nucleotide and amino acid sequences for V_{H} and V_{L} as well as the CDR sequences for the antibodies discloses herein (SEQ ID NOs: 1-90).

| SEQ ID NO: | Antibody | Description | Sequence |
|---|---|---|---|
| SEQ ID NO: 1 | S1scAb06 | Nucleotide | CAGGTGCAGCTGGTGGAGTCTGGGGGAGG |
| | | Sequence of | CGTGGTCCAGCCTGGGAGGTCCCTGAGAC |
| | | V_{H} | TCTCCTGTGCAGCCTCTGGATTCACCTTCA |
| | | | GTAGCTATGGCATGCACTGGGTCCGCCAG |
| | | | GCTCCAGGCAAGGGGCTGGAGTGGGTGGC |
| | | | AGTTATATCATATGATGGAAGTAATAAAT |
| | | | ACTATGCAGACTCCGTGAAGGGCCGATTC |
| | | | ACCATCTCCAGAGACAATTCCAAGAACAC |
| | | | GCTGTATCTGCAAATGAACAGCCTGAGAG |
| | | | CTGAGGACACGGCTGTGTATTACTGTGCG |
| | | | AAATCGCTCCGCAACTCGTTTGACTACTGG |
| | | | GGCCAGGGAACCCTGGTCACCGTCTCCTCA |
| SEQ ID NO: 2 | S1scAb06 | Amino acid | QVQLVESGGGVVQPGRSLRLSCAASGFTFSS |
| | | Sequence of | YGMHWVRQAPGKGLEWVAVISYDGSNKY |
| | | V_{H} | YADSVKGRFTISRDNSKNTLYLQMNSLRAE |
| | | | DTAVYYCAKSLRNSFDYWGQGTLVTVSS |
| SEQ ID NO: 3 | S1scAb06 | Amino acid | GFTFSSYG |
| | | Sequence of | |
| | | V_{H} CDR1 | |
| SEQ ID NO: 4 | S1scAb06 | Amino acid | ISYDGSNK |
| | | Sequence of | |
| | | V_{H} CDR2 | |
| SEQ ID NO: 5 | S1scAb06 | Amino acid | AKSLRNSFDY |
| | | Sequence of | |
| | | V_{H} CDR3 | |
| SEQ ID NO: 6 | S1scAb06 | Nucleotide | GAAATTGTGTTGACGCAGTCTCCAGGCAC |
| | | Sequence of | CCTGTCTTTGTCTCCAGGGGAAAGAGCCA |
| | | V_{L} | CCCTCTCCTGCAGGGCCAGTCAGAGTGTTA |
| | | | GCAGCAACTACTTAGCCTGGTACCAGCAG |
| | | | AAACCTGGCCAGGCTCCCAGGCTCCTCAT |
| | | | CTATGGTGCATCCAGCAGGCCCACTGGCA |
| | | | TCCCAGACAGGTTCAGTGGCAGTGGGTCT |
| | | | GGGACAGACTTCACTCTCACCATCAGCAG |
| | | | ACTGGAGCCTGAAGATTTTGCAGTGTATTA |
| | | | CTGTCAGCAGTATGCTGCCTCACCCCTCAC |
| | | | TTTCGGCGGAGGGACCAAGCTGGAGATCA |
| | | | AA |
| SEQ ID NO: 7 | S1scAb06 | Amino acid | EIVLTQSPGTLSLSPGERATLSCRASQSVSSN |
| | | Sequence of | YLAWYQQKPGQAPRLLIYGASSRPTGIPDRF |
| | | V_{L} | SGSGSGTDFTLTISRLEPEDFAVYYCQQYAA |
| | | | SPLTFGGGTKLEIK |
| SEQ ID NO: 8 | S1scAb06 | Amino acid | QSVSSNY |
| | | Sequence of | |
| | | V_{L} CDR1 | |
| SEQ ID NO: 9 | S1scAb06 | Amino acid | GAS |
| | | Sequence of | |
| | | V_{L} CDR2 | |
| SEQ ID NO: 10 | S1scAb06 | Amino acid | QQYAASPLT |
| | | Sequence of | |
| | | V_{L} CDR3 | |
| SEQ ID NO: 11 | S1scAb11 | Nucleotide | CAGGTGCAGCTGTTGCAGTCTGGGGGAGG |
| | | Sequence of | CGTGGTCCAGCCTGGGAGGTCCCTGAGAC |
| | | V_{H} | TCTCCTGTGCAGCCTCTGGATTCACCTTCA |
| | | | GTAGCTATGGCATGCACTGGGTCCGCCAG |
| | | | GCTCCAGGCAAGGGGCTGGAGTGGGTGGC |
| | | | AGTTATATCATATGATGGAAGTAATAAAT |
| | | | ACTATGCAGACTCCGTGAAGGGCCGATTC |
| | | | ACCATCTCCAGAGACAATTCCAAGAACAC |
| | | | GCTGTATCTGCAAATGAACAGCCTGAGAG |
| | | | CTGAGGACACGGCTGTGTATTACTGTGCG |
| | | | AAAGGCTACGAAAACTACTTTGACTACTG |
| | | | GGGCCAGGGAACCCTGGTCACCGTCTCCT |
| | | | CA |
| SEQ ID NO: 12 | S1scAb11 | Amino acid | QVQLLQSGGGVVQPGRSLRLSCAASGFTFSS |
| | | Sequence of | YGMHWVRQAPGKGLEWVAVISYDGSNKY |
| | | V_{H} | YADSVKGRFTISRDNSKNTLYLQMNSLRAE |
| | | | DTAVYYCAKGYENYFDYWGQGTLVTVSS |
| SEQ ID NO: 13 | S1scAb11 | Amino acid | GFTFSSYG |
| | | Sequence of | |
| | | V_{H} CDR1 | |
| SEQ ID NO: 14 | S1scAb11 | Amino acid | ISYDGSNK |
| | | Sequence of | |
| | | V_{H} CDR2 | |
| SEQ ID NO: 15 | S1scAb11 | Amino acid | AKGYENYFDY |
| | | Sequence of | |
| | | V_{H} CDR3 | |
| SEQ ID NO: 16 | S1scAb11 | Nucleotide | GAAATTGTGCTGACTCAGTCTCCAGACAC |
| | | Sequence of | CCTGTCTTTGTCTCCAGGGGAAAGAGCCA |
| | | V_{L} | CCCTCTCCTGCAGGGCCAGTCAGAGTGTTG |
| | | | CCAGCAACTACTTAGCCTGGTACCAGCAG |
| | | | AAACCTGGCCAGGCTCCCACACTCCTCATC |
| | | | TATAATGCATCCACCAGGGCCACTGGCAT |
| | | | CCCCGACAGGTTCAGTGGCAGTGGGTCTG |
| | | | GGACAGACTTCACTCTCACCATCAGCAGA |
| | | | CTGGAGCCTGAAGATTTTGCAGTGTATTAC |
| | | | TGTCAGCAGTATAGTGCCTCCCCTCTCACT |
| | | | TTCGGCGGAGGGACCAAGGTGGAGATCAAA |
| SEQ ID NO: 17 | S1scAb11 | Amino acid | EIVLTQSPDTLSLSPGERATLSCRASQSVASN |
| | | Sequence of | YLAWYQQKPGQAPTLLIYNASTRATGIPDRF |
| | | V_{L} | SGSGSGTDFTLTISRLEPEDFAVYYCQQYSAS |
| | | | PLTFGGGTKVEIK |
| SEQ ID NO: 18 | S1scAb11 | Amino acid | QSVASNY |
| | | Sequence of | |
| | | V_{L} CDR1 | |
| SEQ ID NO: 19 | S1scAb11 | Amino acid | NAS |
| | | Sequence of | |
| | | V_{L} CDR2 | |
| SEQ ID NO: 20 | S1scAb11 | Amino acid | QQYSASPLT |
| | | Sequence of | |
| | | V_{L} CDR3 | |
| SEQ ID NO: 21 | S2H1 | Nucleotide | CAGGTGCAGCTGTTGCAGTCTGGGGGAGG |
| | | Sequence of | CGTGGTCCAGCCTGGGAGGTCCCTGAGAC |
| | | V_{H} | TCTCCTGTGCAGCCTCTGGATTCACCTTCA |
| | | | GTAGCTATGGCATGCACTGGGTCCGCCAG |
| | | | GCTCCAGGCAAGGGGCTGGAGTGGGTGGC |
| | | | AGTTATATCATATGATGGAAGTAATAAAT |
| | | | ACTATGCAGACTCCGTGAAGGGCCGATTC |
| | | | ACCATCTCCAGAGACAATTCCAAGAACAC |
| | | | GCTGTATCTGCAAATGAACAGCCTGAGAG |
| | | | CTGAGGACACGGCTGTGTATTACTGTGCGT |
| | | | CTCTTTACGAAAACTACTTTTCGCTTTGGG |
| | | | GCCAGGGAACCCTGGTCACCGTCTCCTCA |
| SEQ ID NO: 22 | S2H1 | Amino acid | QVQLLQSGGGVVQPGRSLRLSCAASGFTFSS |
| | | Sequence of | YGMHWVRQAPGKGLEWVAVISYDGSNKY |
| | | V_{H} | YADSVKGRFTISRDNSKNTLYLQMNSLRAE |
| | | | DTAVYYCASLYENYFSLWGQGTLVTVSS |
| SEQ ID NO: 23 | S2H1 | Amino acid | GFTFSSYG |
| | | Sequence of | |
| | | V_{H} CDR1 | |
| SEQ ID NO: 24 | S2H1 | Amino acid | ISYDGSNK |
| | | Sequence of | |
| | | V_{H} CDR2 | |
| SEQ ID NO: 25 | S2H1 | Amino acid | ASLYENYFSL |
| | | Sequence of | |
| | | V_{H} CDR3 | |
| SEQ ID NO: 26 | S2H1 | Nucleotide | GAAATTGTGTTGACGCAGTCTCCAGGCAC |
| | | Sequence of | CCTGTCTTTGTCTCCAGGGGAAAGAGCCA |
| | | V_{L} | CCCTCTCCTGCAGGGCCAGTCAGAGTGTTA |
| | | | GCAGCAACTACTTAGCCTGGTACCAGCAG |
| | | | AAACCTGGCCAGGCTCCCAGGCTCCTCAT |
| | | | CTATGGTGCATCCAGCAGGCCCACTGGCA |
| | | | TCCCAGACAGGTTCAGTGGCAGTGGGTCT |
| | | | GGGACAGACTTCACTCTCACCATCAGCAG |
| | | | ACTGGAGCCTGAAGATTTTGCAGTGTATTA |
| | | | CTGTCAGCAGTATGCTGCCTCACCCCTCAC |
| | | | TTTCGGCGGAGGGACCAAGCTGGAGATCA |
| | | | AA |
| SEQ ID NO: 27 | S2H1 | Amino acid | EIVLTQSPGTLSLSPGERATLSCRASQSVSSN |
| | | Sequence of | YLAWYQQKPGQAPRLLIYGASSRPTGIPDRF |
| | | V_{L} | SGSGSGTDFTLTISRLEPEDFAVYYCQQYAA |
| | | | SPLTFGGGTKLEIK |
| SEQ ID NO: 28 | S2H1 | Amino acid | QSVSSNY |
| | | Sequence of | |
| | | V_{L} CDR1 | |
| SEQ ID NO: 29 | S2H1 | Amino acid | GAS |
| | | Sequence of | |
| | | V_{L} CDR2 | |
| SEQ ID NO: 30 | S2H1 | Amino acid | QQYAASPLT |
| | | Sequence of | |
| | | V_{L} CDR3 | |
| SEQ ID NO: 31 | S2H2 | Nucleotide | CAGGTGCAGCTGTTGCAGTCTGGGGGAGG |
| | | Sequence of | CGTGGTCCAGCCTGGGAGGTCCCTGAGAC |
| | | V_{H} | TCTCCTGTGCAGCCTCTGGATTCACCTTCA |
| | | | GTAGCTATGGCATGCACTGGGTCCGCCAG |
| | | | GCTCCAGGCAAGGGGCTGGAGTGGGTGGC |
| | | | AGTTATATCATATGATGGAAGTAATAAAT |
| | | | ACTATGCAGACTCCGTGAAGGGCCGATTC |
| | | | ACCATCTCCAGAGACAATTCCAAGAACAC |
| | | | GCTGTATCTGCAAATGAACAGCCTGAGAG |
| | | | CTGAGGACACGGCTGTGTATTACTGTGCG |
| | | | ACGTTTCGTGAAAACTACTTTGAGTACTGG |
| | | | GGCCAGGGAACCCTGGTCACCGTCTCCTCA |
| SEQ ID NO: 32 | S2H2 | Amino acid | QVQLLQSGGGVVQPGRSLRLSCAASGFTFSS |
| | | Sequence of | YGMHWVRQAPGKGLEWVAVISYDGSNKY |
| | | V_{H} | YADSVKGRFTISRDNSKNTLYLQMNSLRAE |
| | | | DTAVYYCATFRENYFEYWGQGTLVTVSS |
| SEQ ID NO: 33 | S2H2 | Amino acid | GFTFSSYG |
| | | Sequence of | |
| | | V_{H} CDR1 | |
| SEQ ID NO: 34 | S2H2 | Amino acid | ISYDGSNK |
| | | Sequence of | |
| | | V_{H} CDR2 | |
| SEQ ID NO: 35 | S2H2 | Amino acid | ATFRENYFEY |
| | | Sequence of | |
| | | V_{H} CDR3 | |
| SEQ ID NO: 36 | S2H2 | Nucleotide | GAAATTGTGTTGACGCAGTCTCCAGGCAC |
| | | Sequence of | CCTGTCTTTGTCTCCAGGGGAAAGAGCCA |
| | | V_{L} | CCCTCTCCTGCAGGGCCAGTCAGAGTGTTA |
| | | | GCAGCAACTACTTAGCCTGGTACCAGCAG |
| | | | AAACCTGGCCAGGCTCCCAGGCTCCTCAT |
| | | | CTATGGTGCATCCAGCAGGCCCACTGGCA |
| | | | TCCCAGACAGGTTCAGTGGCAGTGGGTCT |
| | | | GGGACAGACTTCACTCTCACCATCAGCAG |
| | | | ACTGGAGCCTGAAGATTTTGCAGTGTATTA |
| | | | CTGTCAGCAGTATGCTGCCTCACCCCTCAC |
| | | | TTTCGGCGGAGGGACCAAGCTGGAGATCA |
| | | | AA |
| SEQ ID NO: 37 | S2H2 | Amino acid | EIVLTQSPGTLSLSPGERATLSCRASQSVSSN |
| | | Sequence of | YLAWYQQKPGQAPRLLIYGASSRPTGIPDRF |
| | | V_{L} | SGSGSGTDFTLTISRLEPEDFAVYYCQQYAA |
| | | | SPLTFGGGTKLEIK |
| SEQ ID NO: 38 | S2H2 | Amino acid | QSVSSNY |
| | | Sequence of | |
| | | V_{L} CDR1 | |
| SEQ ID NO: 39 | S2H2 | Amino acid | GAS |
| | | Sequence of | |
| | | V_{L} CDR2 | |
| SEQ ID NO: 40 | S2H2 | Amino acid | QQYAASPLT |
| | | Sequence of | |
| | | V_{L} CDR3 | |
| SEQ ID NO: 41 | S2H3 | Nucleotide | CAGGTGCAGCTGTTGCAGTCTGGGGGAGG |
| | | Sequence of | CGTGGTCCAGCCTGGGAGGTCCCTGAGAC |
| | | V_{H} | TCTCCTGTGCAGCCTCTGGATTCACCTTCA |
| | | | GTAGCTATGGCATGCACTGGGTCCGCCAG |
| | | | GCTCCAGGCAAGGGGCTGGAGTGGGTGGC |
| | | | AGTTATATCATATGATGGAAGTAATAAAT |
| | | | ACTATGCAGACTCCGTGAAGGGCCGATTC |
| | | | ACCATCTCCAGAGACAATTCCAAGAACAC |
| | | | GCTGTATCTGCAAATGAACAGCCTGAGAG |
| | | | CTGAGGACACGGCTGTGTATTACTGTGCG |
| | | | GGGGTTAGGGAAAACTACTTTACTTACTG |
| | | | GGGCCAGGGAACCCTGGTCACCGTCTCCT |
| | | | CA |
| SEQ ID NO: 42 | S2H3 | Amino acid | QVQLLQSGGGVVQPGRSLRLSCAASGFTFSS |
| | | Sequence of | YGMHWVRQAPGKGLEWVAVISYDGSNKY |
| | | V_{H} | YADSVKGRFTISRDNSKNTLYLQMNSLRAE |
| | | | DTAVYYCAGVRENYFTYWGQGTLVTVSS |
| SEQ ID NO: 43 | S2H3 | Amino acid | GFTFSSYG |
| | | Sequence of | |
| | | V_{H} CDR1 | |
| SEQ ID NO: 44 | S2H3 | Amino acid | ISYDGSNK |
| | | Sequence of | |
| | | V_{H} CDR2 | |
| SEQ ID NO: 45 | S2H3 | Amino acid | AGVRENYFTY |
| | | Sequence of | |
| | | V_{H} CDR3 | |
| SEQ ID NO: 46 | S2H3 | Nucleotide | GAAATTGTGTTGACGCAGTCTCCAGGCAC |
| | | Sequence of | CCTGTCTTTGTCTCCAGGGGAAAGAGCCA |
| | | V_{L} | CCCTCTCCTGCAGGGCCAGTCAGAGTGTTA |
| | | | GCAGCAACTACTTAGCCTGGTACCAGCAG |
| | | | AAACCTGGCCAGGCTCCCAGGCTCCTCAT |
| | | | CTATGGTGCATCCAGCAGGCCCACTGGCA |
| | | | TCCCAGACAGGTTCAGTGGCAGTGGGTCT |
| | | | GGGACAGACTTCACTCTCACCATCAGCAG |
| | | | ACTGGAGCCTGAAGATTTTGCAGTGTATTA |
| | | | CTGTCAGCAGTATGCTGCCTCACCCCTCAC |
| | | | TTTCGGCGGAGGGACCAAGCTGGAGATCA |
| | | | AA |
| SEQ ID NO: 47 | S2H3 | Amino acid | EIVLTQSPGTLSLSPGERATLSCRASQSVSSN |
| | | Sequence of | YLAWYQQKPGQAPRLLIYGASSRPTGIPDRF |
| | | V_{L} | SGSGSGTDFTLTISRLEPEDFAVYYCQQYAA |
| | | | SPLTFGGGTKLEIK |
| SEQ ID NO: 48 | S2H3 | Amino acid | QSVSSNY |
| | | Sequence of | |
| | | V_{L} CDR1 | |
| SEQ ID NO: 49 | S2H3 | Amino acid | GAS |
| | | Sequence of | |
| | | V_{L} CDR2 | |
| SEQ ID NO: 50 | S2H3 | Amino acid | QQYAASPLT |
| | | Sequence of | |
| | | V_{L} CDR3 | |
| SEQ ID NO: 51 | S2H4 | Nucleotide | CAGGTGCAGCTGTTGCAGTCTGGGGGAGG |
| | | Sequence of | CGTGGTCCAGCCTGGGAGGTCCCTGAGAC |
| | | V_{H} | TCTCCTGTGCAGCCTCTGGATTCACCTTCA |
| | | | GTAGCTATGGCATGCACTGGGTCCGCCAG |
| | | | GCTCCAGGCAAGGGGCTGGAGTGGGTGGC |
| | | | AGTTATATCATATGATGGAAGTAATAAAT |
| | | | ACTATGCAGACTCCGTGAAGGGCCGATTC |
| | | | ACCATCTCCAGAGACAATTCCAAGAACAC |
| | | | GCTGTATCTGCAAATGAACAGCCTGAGAG |
| | | | CTGAGGACACGGCTGTGTATTACTGTGCG |
| | | | GGGGTTAGGGAAAACTACTTTTCTTACTGG |
| | | | GGCCAGGGAACCCTGGTCACCGTCTCCTCA |
| SEQ ID NO: 52 | S2H4 | Amino acid | QVQLLQSGGGVVQPGRSLRLSCAASGFTFSS |
| | | Sequence of | YGMHWVRQAPGKGLEWVAVISYDGSNKY |
| | | V_{H} | YADSVKGRFTISRDNSKNTLYLQMNSLRAE |
| | | | DTAVYYCAGVRENYFSYWGQGTLVTVSS |
| SEQ ID NO: 53 | S2H4 | Amino acid | GFTFSSYG |
| | | Sequence of | |
| | | V_{H} CDR1 | |
| SEQ ID NO: 54 | S2H4 | Amino acid | ISYDGSNK |
| | | Sequence of | |
| | | V_{H} CDR2 | |
| SEQ ID NO: 55 | S2H4 | Amino acid | AGVRENYFSY |
| | | Sequence of | |
| | | V_{H} CDR3 | |
| SEQ ID NO: 56 | S2H4 | Nucleotide | GAAATTGTGTTGACGCAGTCTCCAGGCAC |
| | | Sequence of | CCTGTCTTTGTCTCCAGGGGAAAGAGCCA |
| | | V_{L} | CCCTCTCCTGCAGGGCCAGTCAGAGTGTTA |
| | | | GCAGCAACTACTTAGCCTGGTACCAGCAG |
| | | | AAACCTGGCCAGGCTCCCAGGCTCCTCAT |
| | | | CTATGGTGCATCCAGCAGGCCCACTGGCA |
| | | | TCCCAGACAGGTTCAGTGGCAGTGGGTCT |
| | | | GGGACAGACTTCACTCTCACCATCAGCAG |
| | | | ACTGGAGCCTGAAGATTTTGCAGTGTATTA |
| | | | CTGTCAGCAGTATGCTGCCTCACCCCTCAC |
| | | | TTTCGGCGGAGGGACCAAGCTGGAGATCA |
| | | | AA |
| SEQ ID NO: 57 | S2H4 | Amino acid | EIVLTQSPGTLSLSPGERATLSCRASQSVSSN |
| | | Sequence of | YLAWYQQKPGQAPRLLIYGASSRPTGIPDRF |
| | | V_{L} | SGSGSGTDFTLTISRLEPEDFAVYYCQQYAA |
| | | | SPLTFGGGTKLEIK |
| SEQ ID NO: 58 | S2H4 | Amino acid | QSVSSNY |
| | | Sequence of | |
| | | V_{L} CDR1 | |
| SEQ ID NO: 59 | S2H4 | Amino acid | GAS |
| | | Sequence of | |
| | | V_{L} CDR2 | |
| SEQ ID NO: 60 | S2H4 | Amino acid | QQYAASPLT |
| | | Sequence of | |
| | | V_{L} CDR3 | |
| SEQ ID NO: 61 | S2H5 | Nucleotide | CAGGTGCAGCTGTTGCAGTCTGGGGGAGG |
| | | Sequence of | CGTGGTCCAGCCTGGGAGGTCCCTGAGAC |
| | | V_{H} | TCTCCTGTGCAGCCTCTGGATTCACCTTCA |
| | | | GTAGCTATGGCATGCACTGGGTCCGCCAG |
| | | | GCTCCAGGCAAGGGGCTGGAGTGGGTGGC |
| | | | AGTTATATCATATGATGGAAGTAATAAAT |
| | | | ACTATGCAGACTCCGTGAAGGGCCGATTC |
| | | | ACCATCTCCAGAGACAATTCCAAGAACAC |
| | | | GCTGTATCTGCAAATGAACAGCCTGAGAG |
| | | | CTGAGGACACGGCTGTGTATTACTGTGCGT |
| | | | CTCGTTACGAAAACTACTTTTCTCTGTGGG |
| | | | GCCAGGGAACCCTGGTCACCGTCTCCTCA |
| SEQ ID NO: 62 | S2H5 | Amino acid | QVQLLQSGGGVVQPGRSLRLSCAASGFTFSS |
| | | Sequence of | YGMHWVRQAPGKGLEWVAVISYDGSNKY |
| | | V_{H} | YADSVKGRFTISRDNSKNTLYLQMNSLRAE |
| | | | DTAVYYCASRYENYFSLWGQGTLVTVSS |
| SEQ ID NO: 63 | S2H5 | Amino acid | GFTFSSYG |
| | | Sequence of | |
| | | V_{H} CDR1 | |
| SEQ ID NO: 64 | S2H5 | Amino acid | ISYDGSNK |
| | | Sequence of | |
| | | V_{H} CDR2 | |
| SEQ ID NO: 65 | S2H5 | Amino acid | ASRYENYFSL |
| | | Sequence of | |
| | | V_{H} CDR3 | |
| SEQ ID NO: 66 | S2H5 | Nucleotide | GAAATTGTGTTGACGCAGTCTCCAGGCAC |
| | | Sequence of | CCTGTCTTTGTCTCCAGGGGAAAGAGCCA |
| | | V_{L} | CCCTCTCCTGCAGGGCCAGTCAGAGTGTTA |
| | | | GCAGCAACTACTTAGCCTGGTACCAGCAG |
| | | | AAACCTGGCCAGGCTCCCAGGCTCCTCAT |
| | | | CTATGGTGCATCCAGCAGGCCCACTGGCA |
| | | | TCCCAGACAGGTTCAGTGGCAGTGGGTCT |
| | | | GGGACAGACTTCACTCTCACCATCAGCAG |
| | | | ACTGGAGCCTGAAGATTTTGCAGTGTATTA |
| | | | CTGTCAGCAGTATGCTGCCTCACCCCTCAC |
| | | | TTTCGGCGGAGGGACCAAGCTGGAGATCA |
| | | | AA |
| SEQ ID NO: 67 | S2H5 | Amino acid | EIVLTQSPGTLSLSPGERATLSCRASQSVSSN |
| | | Sequence of | YLAWYQQKPGQAPRLLIYGASSRPTGIPDRF |
| | | V_{L} | SGSGSGTDFTLTISRLEPEDFAVYYCQQYAA |
| | | | SPLTFGGGTKLEIK |
| SEQ ID NO: 68 | S2H5 | Amino acid | QSVSSNY |
| | | Sequence of | |
| | | V_{L} CDR1 | |
| SEQ ID NO: 69 | S2H5 | Amino acid | GAS |
| | | Sequence of | |
| | | V_{L} CDR2 | |
| SEQ ID NO: 70 | S2H5 | Amino acid | QQYAASPLT |
| | | Sequence of | |
| | | V_{L} CDR3 | |
| SEQ ID NO: 71 | S2H6 | Nucleotide | CAGGTGCAGCTGTTGCAGTCTGGGGGAGG |
| | | Sequence of | CGTGGTCCAGCCTGGGAGGTCCCTGAGAC |
| | | V_{H} | TCTCCTGTGCAGCCTCTGGATTCACCTTCA |
| | | | GTAGCTATGGCATGCACTGGGTCCGCCAG |
| | | | GCTCCAGGCAAGGGGCTGGAGTGGGTGGC |
| | | | AGTTATATCATATGATGGAAGTAATAAAT |
| | | | ACTATGCAGACTCCGTGAAGGGCCGATTC |
| | | | ACCATCTCCAGAGACAATTCCAAGAACAC |
| | | | GCTGTATCTGCAAATGAACAGCCTGAGAG |
| | | | CTGAGGACACGGCTGTGTATTACTGTGCGT |
| | | | CTTTTCAGGAAAACTACTTTACGTACTGGG |
| | | | GCCAGGGAACCCTGGTCACCGTCTCCTCA |
| SEQ ID NO: 72 | S2H6 | Amino acid | QVQLLQSGGGVVQPGRSLRLSCAASGFTFSS |
| | | Sequence of | YGMHWVRQAPGKGLEWVAVISYDGSNKY |
| | | V_{H} | YADSVKGRFTISRDNSKNTLYLQMNSLRAE |
| | | | DTAVYYCASFQENYFTYWGQGTLVTVSS |
| SEQ ID NO: 73 | S2H6 | Amino acid | GFTFSSYG |
| | | Sequence of | |
| | | V_{H} CDR1 | |
| SEQ ID NO: 74 | S2H6 | Amino acid | ISYDGSNK |
| | | Sequence of | |
| | | V_{H} CDR2 | |
| SEQ ID NO: 75 | S2H6 | Amino acid | ASFQENYFTY |
| | | Sequence of | |
| | | V_{H} CDR3 | |
| SEQ ID NO: 76 | S2H6 | Nucleotide | GAAATTGTGTTGACGCAGTCTCCAGGCAC |
| | | Sequence of | CCTGTCTTTGTCTCCAGGGGAAAGAGCCA |
| | | V_{L} | CCCTCTCCTGCAGGGCCAGTCAGAGTGTTA |
| | | | GCAGCAACTACTTAGCCTGGTACCAGCAG |
| | | | AAACCTGGCCAGGCTCCCAGGCTCCTCAT |
| | | | CTATGGTGCATCCAGCAGGCCCACTGGCA |
| | | | TCCCAGACAGGTTCAGTGGCAGTGGGTCT |
| | | | GGGACAGACTTCACTCTCACCATCAGCAG |
| | | | ACTGGAGCCTGAAGATTTTGCAGTGTATTA |
| | | | CTGTCAGCAGTATGCTGCCTCACCCCTCAC |
| | | | TTTCGGCGGAGGGACCAAGCTGGAGATCA |
| | | | AA |
| SEQ ID NO: 77 | S2H6 | Amino acid | EIVLTQSPGTLSLSPGERATLSCRASQSVSSN |
| | | Sequence of | YLAWYQQKPGQAPRLLIYGASSRPTGIPDRF |
| | | V_{L} | SGSGSGTDFTLTISRLEPEDFAVYYCQQYAA |
| | | | SPLTFGGGTKLEIK |
| SEQ ID NO: 78 | S2H6 | Amino acid | QSVSSNY |
| | | Sequence of | |
| | | V_{L} CDR1 | |
| SEQ ID NO: 79 | S2H6 | Amino acid | GAS |
| | | Sequence of | |
| | | V_{L} CDR2 | |
| SEQ ID NO: 80 | S2H6 | Amino acid | QQYAASPLT |
| | | Sequence of | |
| | | V_{L} CDR3 | |
| SEQ ID NO: 81 | S2H7 | Nucleotide | CAGGTGCAGCTGTTGCAGTCTGGGGGAGG |
| | | Sequence of | CGTGGTCCAGCCTGGGAGGTCCCTGAGAC |
| | | V_{H} | TCTCCTGTGCAGCCTCTGGATTCACCTTCA |
| | | | GTAGCTATGGCATGCACTGGGTCCGCCAG |
| | | | GCTCCAGGCAAGGGGCTGGAGTGGGTGGC |
| | | | AGTTATATCATATGATGGAAGTAATAAAT |
| | | | ACTATGCAGACTCCGTGAAGGGCCGATTC |
| | | | ACCATCTCCAGAGACAATTCCAAGAACAC |
| | | | GCTGTATCTGCAAATGAACAGCCTGAGAG |
| | | | CTGAGGACACGGCTGTGTATTACTGTGCG |
| | | | ACTCGGTACGAAAACTACTTTTCTACGTGG |
| | | | GGCCAGGGAACCCTGGTCACCGTCTCCTCA |
| SEQ ID NO: 82 | S2H7 | Amino acid | QVQLLQSGGGVVQPGRSLRLSCAASGFTFSS |
| | | Sequence of | YGMHWVRQAPGKGLEWVAVISYDGSNKY |
| | | V_{H} | YADSVKGRFTISRDNSKNTLYLQMNSLRAE |
| | | | DTAVYYCATRYENYFSTWGQGTLVTVSS |
| SEQ ID NO: 83 | S2H7 | Amino acid | GFTFSSYG |
| | | Sequence of | |
| | | V_{H} CDR1 | |
| SEQ ID NO: 84 | S2H7 | Amino acid | ISYDGSNK |
| | | Sequence of | |
| | | V_{H} CDR2 | |
| SEQ ID NO: 85 | S2H7 | Amino acid | ATRYENYFST |
| | | Sequence of | |
| | | V_{H} CDR3 | |
| SEQ ID NO: 86 | S2H7 | Nucleotide | GAAATTGTGTTGACGCAGTCTCCAGGCAC |
| | | Sequence of | CCTGTCTTTGTCTCCAGGGGAAAGAGCCA |
| | | V_{L} | CCCTCTCCTGCAGGGCCAGTCAGAGTGTTA |
| | | | GCAGCAACTACTTAGCCTGGTACCAGCAG |
| | | | AAACCTGGCCAGGCTCCCAGGCTCCTCAT |
| | | | CTATGGTGCATCCAGCAGGCCCACTGGCA |
| | | | TCCCAGACAGGTTCAGTGGCAGTGGGTCT |
| | | | GGGACAGACTTCACTCTCACCATCAGCAG |
| | | | ACTGGAGCCTGAAGATTTTGCAGTGTATTA |
| | | | CTGTCAGCAGTATGCTGCCTCACCCCTCAC |
| | | | TTTCGGCGGAGGGACCAAGCTGGAGATCA |
| | | | AA |
| SEQ ID NO: 87 | S2H7 | Amino acid | EIVLTQSPGTLSLSPGERATLSCRASQSVSSN |
| | | Sequence of | YLAWYQQKPGQAPRLLIYGASSRPTGIPDRF |
| | | V_{L} | SGSGSGTDFTLTISRLEPEDFAVYYCQQYAA |
| | | | SPLTFGGGTKLEIK |
| SEQ ID NO: 88 | S2H7 | Amino acid | QSVSSNY |
| | | Sequence of | |
| | | V_{L} CDR1 | |
| SEQ ID NO: 89 | S2H7 | Amino acid | GAS |
| | | Sequence of | |
| | | V_{L} CDR2 | |
| SEQ ID NO: 90 | S2H7 | Amino acid | QQYAASPLT |
| | | Sequence of | |
| | | V_{L} CDR3 | |

### Example 3: The Anti-Leptin Receptor Antibodies of the Present Technology are Leptin Receptor Agonists

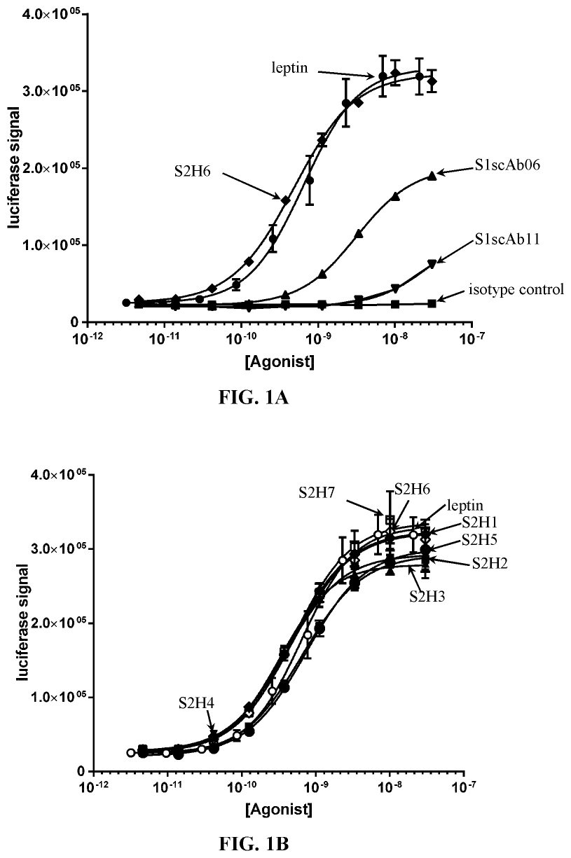

Leptin activates the Stat1 and Stat3 signaling pathways, and modulates gene expression via the SIE element (sis-inducible element), which is a canonical STAT binding sequence. See, e.g., Bendinelli et al., Mol Cell Endocrinol. 168(1-2):11-20 (2000). To understand whether the anti-leptin receptor antibodies disclosed herein modulate gene expression via the SIE element, the SIE-luciferase reporter was used. The SIE-luciferase reporter cells were diluted to 0.4 million cells/ml and seeded into TC-treated white opaque 96 plate (50 μl/well). Leptin or the anti-leptin receptor antibodies S1scAb06, S1scAb11, and S2H6 were serially diluted and added to the cells (50 μl/well). The cells were cultured for 6-8 hr. Luciferase assay substrate was to the cells and luminescence measured using a microplate reader. As shown in FIG. 1A, leptin, and the S1scAb11, S1scAb6, and S2H6 antibodies induced luciferase expression. The isotype control antibody, which serves as a negative control for leptin receptor binding, did not induce a detectable expression of the SIE-luciferase reporter (FIG. 1A). As shown in FIG. 1A, the S1scAb11 and S1scAb6 antibodies, which were selected in the first round of selection, induced the SIE-luciferase reporter expression at a higher concentration compared to leptin. The S2H6 antibody, which was selected in the second round of selection, induced the SIE-luciferase reporter expression at a lower concentration compared to S1scAb11 and S1scAb6 antibodies. Accordingly, S1scAb11, S1scAb6, and S2H6 antibodies bind to leptin receptor and activate downstream signal transduction. Therefore, these data indicate that the S1scAb11, S1scAb6, and S2H6 antibodies are leptin receptor agonists. The Table below shows the EC₅₀ values (M) for activation of leptin receptor as measured by the luciferase assay.

| SC2H6 | S1scAb06 | S1scAb11 | hleptin |
|---|---|---|---|
| 4.78E−10 | 3.18E−9 | ND | 6.40E−10 |

The anti-leptin receptor antibodies S2H1, S2H2, S2H3, S2H4, S2H5, S2H6, and S2H7, which was selected in the second round of selection, were also compared with leptin for modulation of gene expression via the SIE element in the SIE-luciferase reporter cells. As shown in FIG. 1B, each of S2H1, S2H2, S2H3, S2H4, S2H5, S2H6, and S2H7 induced the SIE-luciferase reporter expression. The Table below shows the EC₅₀ values (M) for activation of leptin receptor as measured by the luciferase assay.

| SC2H1 | SC2H2 | SC2H3 | SC2H4 | SC2H5 | SC2H6 | SC2H7 |
|---|---|---|---|---|---|---|
| 4.53E−010 | 6.52E−010 | 3.28E−010 | 3.87E−010 | 7.38E−010 | 4.78E−010 | 4.94E−010 |

These results demonstrate that the anti-leptin receptor antibodies of the present technology are leptin receptor agonists, and thus useful in methods for treating obesity, leptin deficiency, leptin resistance, and/or hypoleptinemia.

### Example 4: The Anti-Leptin Receptor Antibodies of the Present Technology Promote the Growth of Leptin-Dependent Cells

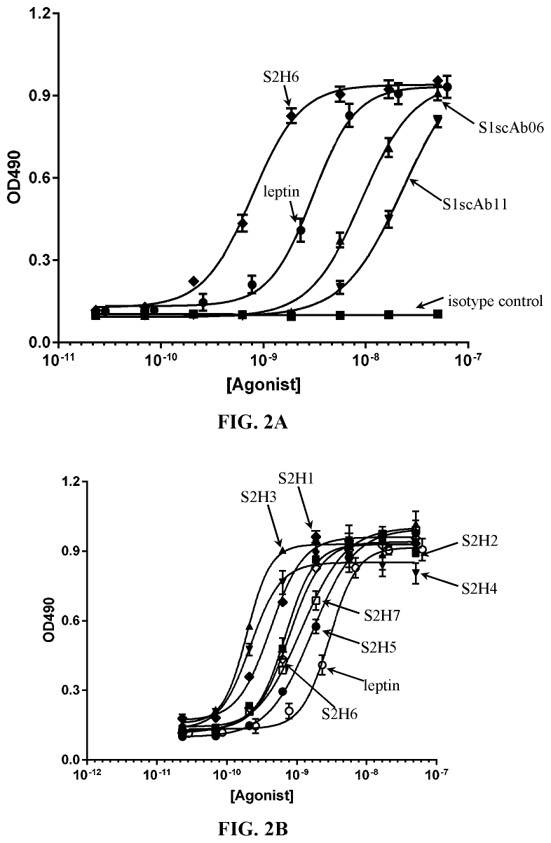

The leptin-dependent Ba/F3-lepR reporter cells were cultured in RPMI 1640 media supplemented with leptin at a concentration of 2 ng/ml. The cells were washed with phosphate-buffered saline (PBS) three times, diluted to 0.2 million cells/ml, and seeded into a 96-well plate (50 μl/well). Leptin or the anti-leptin receptor antibodies S1scAb06, S1scAb11, and S2H6 were serially diluted and added to the cells (50 μl/well). Cells were cultured at 37° C. for another 72 hours. To detect proliferation, CellTiter 96 AQueous One Solution Reagent was added to wells carrying the cells (20 μl/well) and incubated for 2 hr at 37° C. Absorbance at 490 nm was recorded with a microplate reader to measure the level of cell proliferation. As shown in FIG. 2A, the anti-leptin receptor antibodies S1scAb06, S1scAb11, and S2H6 supported the growth of the leptin-dependent Ba/F3-lepR reporter cells. An isotype control antibody, which was used as a negative control, did not promote the proliferation of the leptin-dependent cells (FIG. 2A). As shown in FIG. 2A, he S2H6 antibody, which was obtained after the second round of selection, promoted the growth of the leptin-dependent cells more potently compared to leptin. The Table below compares the EC₅₀ values (M) for activation of leptin receptor as measured by the luciferase assay and the cell proliferation assay.

| EC₅₀(M) | SC2H6 | S1scAb06 | S1scAb11 | hleptin |
|---|---|---|---|---|
| Luciferase assay | 4.78E−10 | 3.18E−9 | ND | 6.40E−10 |
| Cell proliferation assay | 7.73E−10 | 9.14E−9 | 2.30E−8 | 3.01E−9 |

The anti-leptin receptor antibodies S2H1, S2H2, S2H3, S2H4, S2H5, S2H6, and S2H7, which was obtained after the second round of selection, were also compared with leptin for promoting growth of the leptin-dependent Ba/F3-lepR reporter cells. As shown in FIG. 2B, each of S2H1, S2H2, S2H3, S2H4, S2H5, S2H6, and S2H7 promoted the growth of the leptin-dependent cells more potently compared to leptin. The Table below compares the EC₅₀ values (M) for activation of leptin receptor as measured by the luciferase assay and the cell proliferation assay.

| EC₅₀(M) | SC2H1 | SC2H2 | SC2H3 | SC2H4 | SC2H5 | SC2H6 | SC2H7 |
|---|---|---|---|---|---|---|---|
| Luciferase assay | 4.53E−010 | 6.52E−010 | 3.28E−010 | 3.87E−010 | 7.38E−010 | 4.78E−010 | 4.94E−010 |
| Cell proliferation assay | 4.19E−010 | 6.87E−010 | 1.93E−010 | 2.17E−010 | 1.63E−009 | 7.73E−010 | 1.15E−009 |

These results demonstrate that the anti-leptin receptor antibodies of the present technology are leptin receptor agonists, and thus useful in methods for treating obesity, leptin deficiency, leptin resistance, and/or hypoleptinemia.

### Example 5: The Anti-Leptin Receptor Antibodies of the Present Technology are Effective in Mouse Model of Obesity

To evaluate therapeutic effect of the anti-leptin receptor antibodies of the present technology, their effect on a mouse model of obesity was experimentally determined. The mouse model of obesity used for this study was the leptin-deficient (ob/ob) mice. Six-week old female ob/ob mice were maintained in a room with a 12 hour light/dark cycle and provided chow and water ad libitum. Body weight and food intake were monitored daily for 3-4 days prior to the starting dosing and the mice were randomly sorted into three treatment groups: vehicle (PBS), leptin and S2H6 antibody. Mice were injected subcutaneously with the vehicle (twice daily), leptin (0.5 mg/kg, twice daily) and S2H6 (5 mg/kg, once every other day) for two weeks (n=8). The vehicle-treated group served as a negative control for lack of any treatment. The leptin-treated group served as a positive control for reduction of obesity. Body weights and food intake were recorded daily.

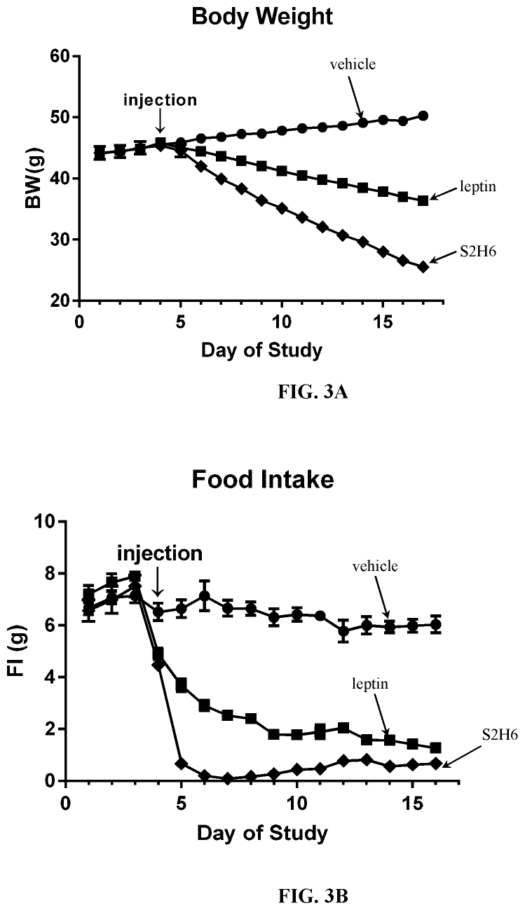

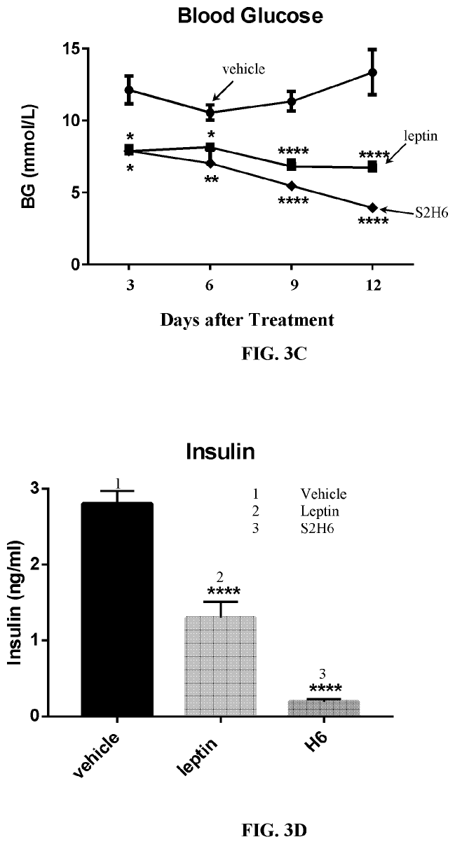

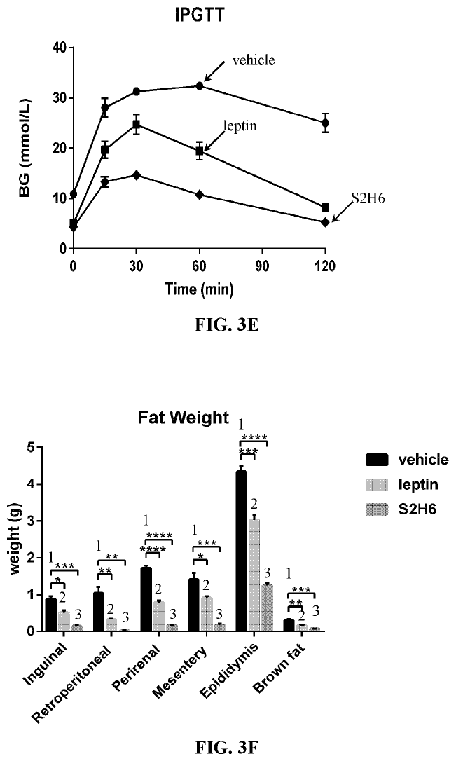

The body weights of vehicle-treated group were measured every day. As shown in FIG. 3A, the body weights of the vehicle-treated group increased during course of the experiment. The leptin-treated, and the S2H6-treated groups showed a reduction in the body weight compared to the vehicle-treated group (FIG. 3A). As apparent from FIG. 3A, the extent of reduction of body weight was more than that observed in the leptin-treated group. The Table below shows body weights of the animals after two weeks of treatment. The reduction in body weight induced by S2H6 treatment was statistically significant as calculated by Student's t test (P<0.0001).

| | Body weights after two weeks of treatment |
|---|---|
| Treatment | (Values shown are mean ± SEM) |
| Vehicle only (n = 8) | 50.30 ± 0.49 g |
| Leptin (n = 8) | 36.39 ± 0.61 g |
| S2H6 (n = 8) | 25.55 ± 0.78 g |

Food Intake was recorded daily during the course of experiment. As shown in FIG. 3B, the food intake by the vehicle-treated group remained unchanged compared to compared to that prior to the starting dosing. The leptin-treated and S2H6-treated groups exhibited a reduction in the food intake compared to the vehicle-treated group (FIG. 3B). As shown in FIG. 3B, S2H6-treated group presented a more reduction in the food intake compared to the leptin-treated group.

Blood glucose was measured twice a week. The blood glucose in the vehicle-treated group remained essentially unchanged during the course of experiment (FIG. 3C). As shown in FIG. 3C, the leptin-treated and S2H6-treated groups exhibited a very significant reduction in the blood glucose compared to the vehicle-treated group. The S2H6-treated group presented a more significant reduction in the blood glucose compared to the leptin-treated group (see second group in FIG. 3C). This difference between blood glucose levels of leptin-treated and S2H6-treated groups was statistically significant (p<0.01) on day 12 after antibody treatment (the last time points shown in FIG. 3C).

After two weeks of dosing, the mice were fasted for 16 h, and blood insulin concentration was measured. As shown in FIG. 3D, the leptin-treated and S2H6-treated groups exhibited a very significant reduction in the blood insulin levels compared to the vehicle-treated group.

The fasted mice were subjected to an intra-peritoneal glucose tolerance test (IPGTT). As shown in FIG. 3E, the leptin-treated and S2H6-treated groups exhibited a reduction in the blood glucose during the IPGTT compared to the vehicle-treated group. The S2H6-treated group showed a more significant reduction in the blood glucose compared to the leptin-treated group (FIG. 3E).

Finally, the mice were sacrificed and adipose tissue from different locations was extracted and weighed. As shown in FIG. 3F, the leptin-treated and S2H6-treated groups exhibited a reduction in the adipose tissue levels during the IPGTT compared to the vehicle-treated group. The S2H6-treated group showed a more significant reduction in the adipose tissue compared to the leptin-treated group (FIG. 3F).

These results demonstrate that the anti-leptin receptor antibodies of the present technology are leptin receptor agonists, and thus useful in methods for treating obesity, leptin deficiency, diabetes, leptin resistance, and/or hypoleptinemia.

### Example 6: The Anti-Leptin Receptor Antibodies of the Present Technology can Compete with Leptin for Occupancy of the Leptin Receptor

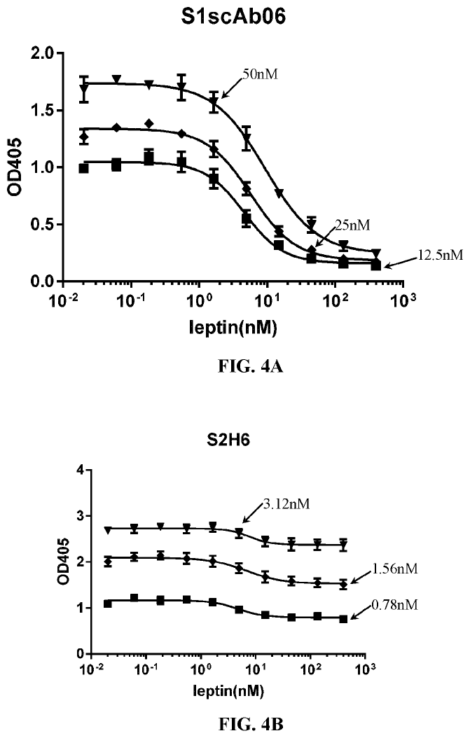

Whether the anti-leptin receptor antibodies of the present technology can compete with leptin for binding to the human leptin receptor was explored. Towards that goal, microplates were coated with the extracellular domain of the human leptin receptor, and increasing concentrations leptin (shown on the X-axis of FIGS. 4A-4B) were added to the microplates along with the indicated fixed antibody concentrations to set up a competition for occupancy of the human leptin receptor. A secondary antibody was used to detect binding of the antibody. As shown in FIG. 4A, leptin could compete with S1scAb06 antibody, which was obtained after the first round of selection, with IC₅₀ for inhibition of binding of S1scAb06 of 6.55 nM. However, as shown in FIG. 4B, leptin could compete with S2H6, which was obtained after the second round of selection. This was consistent with higher affinity of S2H6 to leptin receptor compared to leptin as disclosed herein.

### Example 7: The Affinity of Anti-Leptin Receptor Antibodies of the Present Technology to the Leptin Receptor

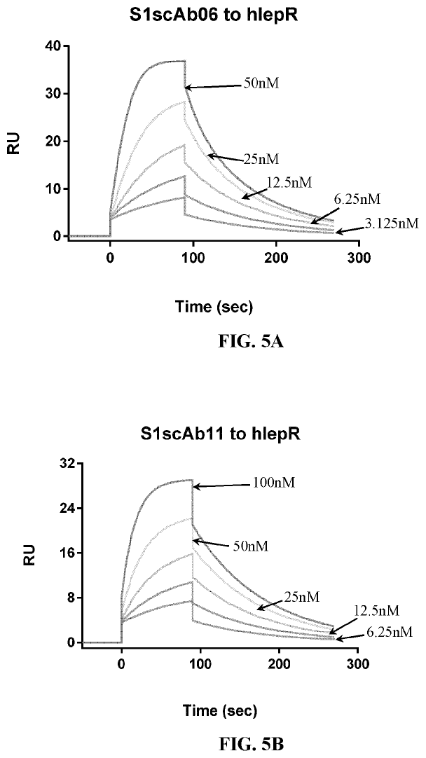

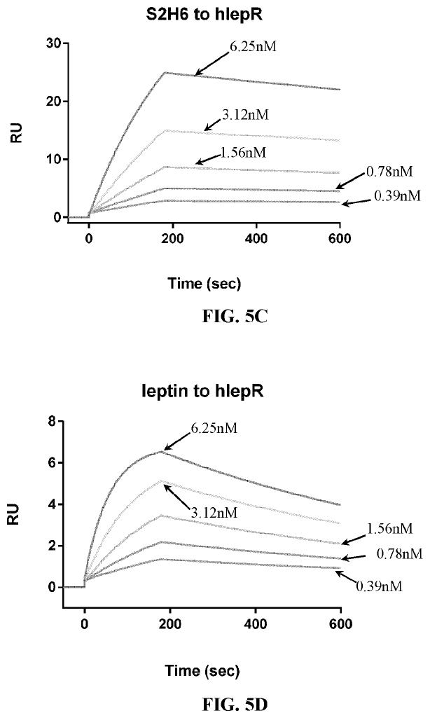

Surface plasmon resonance (SPR) was used for accurate determination of the binding parameters of the anti-leptin receptor antibodies of the present technology. The SPR binding assays were performed using the Biacore T200™ (GE Healthcare). Briefly, the recombinant extracellular domain of leptin receptor having a His-tag was immobilized on the surface of Series S Sensor CM5 chip (GE Healthcare) through Amine Coupling Kit (GE Healthcare). Leptin and different agonist antibodies were diluted serially as analyte. All manipulations were performed as described in the user guide of the manufacturer. The analysis of the results were processed in BIA evaluation Software™. As shown in FIGS. 5A-5D, S1scAb06, S1scAb11, S2H6, and leptin bound to the extracellular domain of leptin receptor. The Table below shows K_{D}, Kₒₙ and K_{off} values of the binding.

| | leptin | S1scAb06 | S1scAb11 | S2H6 |
|---|---|---|---|---|
| K_{D}(M) | 5.04E−10 | 7.47E−9 | 2.35E−8 | 3.90E−10 |
| Kₒₙ(1/M · s) | 2.59E+6 | 6.91E+6 | 4.72E+5 | 7.86E+5 |
| K_{off}(1/s) | 1.30E−3 | 5.16E−2 | 1.11E−2 | 3.08E−4 |

The binding parameters of the anti-leptin receptor antibodies S2H1, S2H2, S2H3, S2H4, S2H5, S2H6, and S2H7 were also determined using SPR. The Table below shows K_{D}, Kₒₙ and K_{off} values of the binding of S2H1, 52H2, S2H3, S2H4, S2H5, S2H6, and S2H7 to the extracellular domain of leptin receptor.

| | S2H1 | S2H2 | S2H3 | S2H4 | S2H5 | S2H6 | S2H7 |
|---|---|---|---|---|---|---|---|
| KD (M) | 5.785E−10 | 3.549E−10 | 4.291E−10 | 5.086E−10 | 6.258E−10 | 3.904E−10 | 6.388E−10 |
| Kon (1/M · s) | 4.678E+5 | 3.362E+5 | 4.663E+5 | 4.768E+5 | 2.618E+5 | 7.86E+5 | 2.676E+5 |
| Koff (1/s) | 2.706E−4 | 1.193E−4 | 2.000E−4 | 2.425E−4 | 1.638E−4 | 3.08E−4 | 1.709E−4 |

### Example 8: The Anti-Leptin Receptor Antibodies of the Present Technology can Activate the Mutant Human Leptin Receptors that are Defective or Impaired in Signaling

Many leptin receptor mutants have been identified that exhibit defective or impaired in leptin-binding capability or leptin-mediated signaling. For example, the LEPR-A409E mutant, which was originally identified as a monogenic cause of early onset obesity, is a signaling-defective mutant leptin receptor that does not transduce leptin signals to STATS. The L372A mutant is also a leptin signaling-defective mutant. The L505/506S mutant is defective in leptin signaling because when leucine is substituted with serine, leptin cannot bind to the receptor.

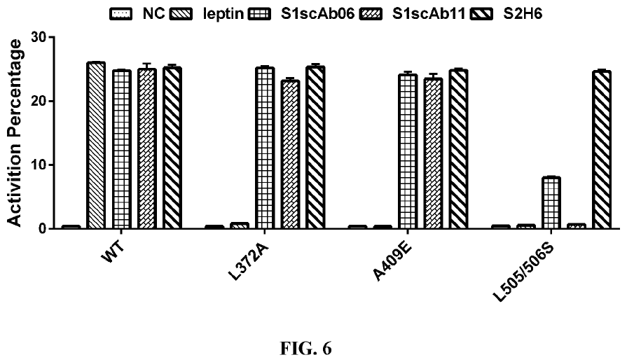

To understand whether the anti-leptin receptor antibodies of the present technology, can activate these mutant receptors, the leptin receptor mutants with signaling deficiency, including L372A, A409E, L505/506S, were constructed. DNA mutagenesis was performed using standard protocols, and all DNA constructs were verified by DNA sequencing. The mutants were transiently transfected in SIE-GFP reporter cells. After 24 h cultivation, leptin and different agonist antibodies were added to the cells for a 8-hr stimulation. Cells harboring wild type (WT) leptin receptor were used as a positive control for signaling proficiency. Vehicle alone was used as a negative control (NC) for the ability to activate leptin receptor. GFP expression was analyzed using flow cytometer and indicated as the leptin signaling activation. S1scAb06, S1scAb11, S2H6 and leptin were all able activate GFP expression by the WT leptin receptor (FIG. 6). Leptin was not able to activate GFP expression by the L372A, A409E, and L505/506S mutants (FIG. 6). In contrast, as shown in FIG. 6, the S1scAb06, S1scAb11, and S2H6 antibodies were able to activate GFP expression by the L372A, A409E, and L505/506S mutants. The S2H6 antibody activated GFP expression by the L505/506S mutant more potently than the S1scAb06, and S1scAb11 antibodies.

Specifically, these results show that the anti-leptin receptor antibodies of the present technology can activate leptin receptor mutants that are defective or impaired in leptin-binding or leptin-mediated signaling. Accordingly, these results demonstrate that the anti-leptin receptor antibodies of the present technology are leptin receptor agonists, and thus useful for treating obesity, leptin deficiency, leptin resistance, and/or hypoleptinemia.

### Example 9: Identification of the Epitope of the Antibodies of the Present Technology

The following six small subdomains of extracellular domain of leptin receptor were expressed and purified: N terminal domain (NTD), first cytokine receptor homology domain (CRH1), an immunoglobulin-like domain (IgD), a second CRH domain (CRH2) and fibronectin type III domains (FNIII). Sequences of these domains are shown in the table below.

| Subdomain of the | |
|---|---|
| LEPR Extracellular | |
| Domain | Sequence |
| NTD F22-Q121 | FNLSYPITPWRFKLSCMPPNSTYDYFLLPAGLSKNTSNSNG |
| (SEQ ID NO: 103) | HYETAVEPKFNSSGTHFSNLSKTTFHCCFRSEQDRNCSLCA |
| | DNIEGKTFVSTVNSLVFQ |
| CRH1 Q122-V333 | QIDANWNIQCWLKGDLKLFICYVESLFKNLFRNYNYKVHL |
| (SEQ ID NO: 104) | LYVLPEVLEDSPLVPQKGSFQMVHCNCSVHECCECLVPVPT |
| | AKLNDTLLMCLKITSGGVIFQSPLMSVQPINMVKPDPPLGL |
| | HMEITDDGNLKISWSSPPLVPFPLQYQVKYSENSTTVIREAD |
| | KIVSATSLLVDSILPGSSYEVQVRGKRLDGPGIWSDWSTPR |
| | VFTTQDV |
| IgD I334-V427 | IYFPPKILTSVGSNVSFHCIYKKENKIVPSKEIVWWMNLAEK |
| (SEQ ID NO: 105) | IPQSQYDVVSDHVSKVTFFNLNETKPRGKFTYDAVYCCNE |
| | HECHHRYAELYV |
| CRH2 I428-D635 | IDVNINISCETDGYLTKMTCRWSTSTIQSLAESTLQLRYHRS |
| (SEQ ID NO: 106) | SLYCSDIPSIFIPISEPKDCYLQSDGFYECIFQPIFLLSGYTMWI |
| | RINHSLGSLDSPPTCVLPDSVVKPLPPSSVKAEITINIGLLKIS |
| | WEKPVFPENNLQFQIRYGLSGKEVQWKMYEVYDAKSKSV |
| | SLPVPDLCAVYAVQVRCKRLDGLGYWSNWSNPAYTVVMD |
| FNIII I636-D839 | IKVPMRGPEFWRIINGDTMKKEKNVTLLWKPLMKNDSLCS |
| (SEQ ID NO: 107) | VQRYVINHHTSCNGTWSEDVGNHTKFTFLWTEQAHTVTVL |
| | AINSIGASVANFNLTFSWPMSKVNIVQSLSAYPLNSSCVIVS |
| | WILSPSDYKLMYFIIEWKNLNEDGEIKWLRISSSVKKYYIHD |
| | HFIPIEKYQFSLYPIFMEGVGKPKIINSFTQDDIEKHQSD |

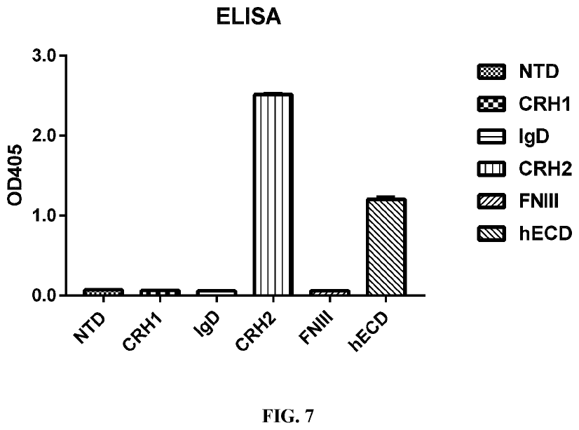

To test ability if the antibody to bind these domains, these subdomain were used in an ELISA-based biding assay. The full length extracellular domain of human leptin receptor (hECD) was used as a positive control. As shown in FIG. 7, only the hECD and CRH2 were able to bind to the S2H6 antibody, indicating that the epitope of S2H6 is located in the CRH2 domain.

To identify the epitope, the S2H6 antibody was cross-linked to the extracellular domain of human leptin receptor using disuccinimidyl sulfoxide (DSSO). Following protease digestion, peptides bearing the crosslink were identified using mass spectrometry. These experiments identified the following three small peptide fragments:

(SEQ ID NO: 100)
Peptide 01. AVQVRC[K]RL
(SEQ ID NO: 101)
Peptide 02. DA[K]SKSVSLPVPDLCAVY
(SEQ ID NO: 102)
Peptide 03. E[K]PVFPENNLQF

[K] indicates the putative site of cross-linking with DSSO. These data indicate that the S2H6 antibody binds to an epitope distributed over these three peptides, suggesting that the S2H6 antibody binds a conformational epitope.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as were apparent to those skilled in the art. Functionally equivalent methods and apparatuses within the scope of the present technology, in addition to those enumerated herein, were apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As were understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as were understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. An anti-leptin receptor antibody, or antigen binding fragment thereof comprising a heavy chain immunoglobulin variable domain (V_{H}) and a light chain immunoglobulin variable domain (V_{L}),
wherein the V_{H} comprises a V_{H}-CDR1 sequence selected from the group consisting of: SEQ ID NOs: 3, 13, 23, 33, 43, 53, 63, 73, and 83; a V_{H}-CDR2 sequence of selected from the group consisting of: SEQ ID NOs: 4, 14, 24, 34, 44, 54, 64, 74, and 84; and a V_{H}-CDR3 sequence selected from the group consisting of: SEQ ID NOs: 5, 15, 25, 35, 45, 55, 65, 75, and 85; and
the V_{L} comprises an amino acid sequence selected from the group consisting of: a V_{L}-CDR1 sequence selected from the group consisting of: SEQ ID NOs: 8, 18, 28, 38, 48, 58, 68, 78, and 88; a V_{L}-CDR2 sequence of selected from the group consisting of: SEQ ID NOs: 9, 19, 29, 39, 49, 59, 69, 79, and 89; and a V_{H}-CDR3 sequence selected from the group consisting of: SEQ ID NOs: 10, 20, 30, 40, 50, 60, 70, 80, and 90.

2. An anti-leptin receptor antibody or antigen binding fragment thereof comprising
a V_{H} amino acid sequence comprising SEQ ID NO: 2, SEQ ID NO: 12, SEQ ID NO: 22, SEQ ID NO: 32, SEQ ID NO: 42, SEQ ID NO: 52, SEQ ID NO: 62, SEQ ID NO: 72, SEQ ID NO: 82, or a variant thereof having one or more conservative amino acid substitutions and
a V_{L} amino acid sequence comprising SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87 or a variant thereof having one or more conservative amino acid substitutions.

3. The anti-leptin receptor antibody or antigen binding fragment of claim 2, comprising a V_{H} amino acid sequence and a V_{L} amino acid sequence selected from the group consisting of:
SEQ ID NO: 2 and SEQ ID NO: 7 (S1scAb06);
SEQ ID NO: 12 and SEQ ID NO: 17 (S1scAb11);
SEQ ID NO: 22 and SEQ ID NO: 27 (S2H1);
SEQ ID NO: 32 and SEQ ID NO: 37 (S2H2);
SEQ ID NO: 42 and SEQ ID NO: 47 (S2H3);
SEQ ID NO: 52 and SEQ ID NO: 57 (S2H4);
SEQ ID NO: 62 and SEQ ID NO: 67 (S2H5);
SEQ ID NO: 72 and SEQ ID NO: 77 (S2H6); and
SEQ ID NO: 82 and SEQ ID NO: 87 (S2H7), respectively.

4. An anti-leptin receptor antibody or antigen binding fragment of claim 1 comprising
(a) a light chain immunoglobulin variable domain sequence that is at least 80%, at least 85%, at least 90%, at least 95%, or at least 99% identical to the light chain immunoglobulin variable domain sequence of any one of SEQ ID NOs: 7, 17, 27, 37, 47, 57, 67, 77, or 87; and
(b) a heavy chain immunoglobulin variable domain sequence (V_{H}) that is at least 80%, at least 85%, at least 90%, at least 95%, or at least 99% identical to the heavy chain immunoglobulin variable domain sequence present in any one of SEQ ID NOs: 2, 12, 22, 32, 42, 52, 62, 72 or 82.

5. The anti-leptin receptor antibody or antigen binding fragment of claim 1, further comprising a Fc domain of an isotype selected from the group consisting of IgG1, IgG2, IgG3, IgG4, IgA1, IgA2, IgM, IgD, and IgE.

6. The anti-leptin receptor antigen binding fragment of claim 1, wherein the antigen binding fragment is selected from the group consisting of Fab, F(ab′)2, Fab′, scFᵥ, and Fᵥ.

7. The anti-leptin receptor antibody of claim 1, wherein the anti-leptin receptor antibody is a monoclonal antibody, a chimeric antibody, a humanized antibody, or a bispecific antibody.

8. The anti-leptin receptor antibody or antigen binding fragment of claim 1, wherein the anti-leptin receptor antibody or antigen binding fragment binds to the CRH2 domain of human leptin receptor.

9. The anti-leptin receptor antibody or antigen binding fragment of claim 1, wherein anti-leptin receptor antibody or antigen binding fragment binds to a conformational epitope.

10. A nucleic acid sequence encoding the antibody or antigen binding fragment of claim 1.

11. The nucleic acid sequence of claim 10 selected from the group consisting of SEQ ID NOs: 1, 6, 11, 16, 21, 26, 31, 36, 41, 46, 51, 56, 61, 66, 71, 76, 81, and 86.

12. A host cell or an expression vector expressing the nucleic acid of claim 10.

13. A composition comprising the anti-leptin receptor antibody or antigen binding fragment of claim 1.

14. A kit comprising the antibody or antigen binding fragment of claim 1 and instructions for use.

15. The kit of claim 14, wherein the antibody or antigen binding fragment is coupled to at least one detectable label selected from the group consisting of a radioactive label, a fluorescent label, and a chromogenic label.

16. A method for detecting leptin receptor in a biological sample comprising
contacting the biological sample with the antibody or antigen binding fragment of claim 1, wherein the antibody or antigen binding fragment is conjugated to a detectable label; and
detecting the levels of the detectable label in the biological sample.

17. A method for treating a disorder associated with or caused by leptin deficiency or hypoleptinemia, leptin resistance, or leptin receptor mutations causing defective or impaired leptin signaling in a subject in need thereof, comprising: administering to the subject a therapeutically effective amount of the antibody or antigen binding fragment of claim 1.

18. A method for alleviating one or more symptoms of a disorder associated with or caused by leptin deficiency or hypoleptinemia, leptin resistance, or leptin receptor mutations causing defective or impaired leptin signaling in a subject in need thereof, comprising: administering to the subject a therapeutically effective amount of the antibody or antigen binding fragment of claim 1.

19. The method of claim 18, wherein the one or more symptoms comprise increased body weight, increased food intake, increased blood glucose levels, decreased insulin levels, and/or decreased glucose tolerance.

20. The method of claim 17, wherein the disorder is obesity.


SEQUENCE LISTING (107 sequences)

SEQ ID NO: 1 - DNA, 351 bp, artificial sequence
  Nucleotide sequence of VH of S1scAb06
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaatcgctccgcaactcgtttgactactggggccagggaaccctggtcaccgtctcctca

SEQ ID NO: 2 - protein, 117 aa, artificial sequence
  Amino acid Sequence of VH of S1scAb06
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKSLRNSFDYWGQGTLVTVSS

SEQ ID NO: 3 - protein, 8 aa, artificial sequence
  Amino acid Sequence of VH CDR1 of S1scAb06
GFTFSSYG

SEQ ID NO: 4 - protein, 8 aa, artificial sequence
  Amino acid Sequence of VH CDR2 of S1scAb06
ISYDGSNK

SEQ ID NO: 5 - protein, 10 aa, artificial sequence
  Amino acid Sequence of VH CDR3 of S1scAb06
AKSLRNSFDY

SEQ ID NO: 6 - DNA, 324 bp, artificial sequence
  Nucleotide Sequence of VL  of S1scAb06
gaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccagcaggcccactggcatcccagacaggttcagtggcagtgggtctgggacagacttcactctcaccatcagcagactggagcctgaagattttgcagtgtattactgtcagcagtatgctgcctcacccctcactttcggcggagggaccaagctggagatcaaa

SEQ ID NO: 7 - protein, 108 aa, artificial sequence
  Amino acid Sequence of VL of S1scAb06
EIVLTQSPGTLSLSPGERATLSCRASQSVSSNYLAWYQQKPGQAPRLLIYGASSRPTGIPDRFSGSGSGTDFTLTISRLEPEDFAVYYCQQYAASPLTFGGGTKLEIK

SEQ ID NO: 8 - protein, 7 aa, artificial sequence
  Amino acid Sequence of VL CDR1 of S1scAb06
QSVSSNY

SEQ ID NO: 9 - protein, 3 aa, artificial sequence
  Amino acid Sequence of VL CDR2 of S1scAb06
GAS

SEQ ID NO: 10 - protein, 9 aa, artificial sequence
  Amino acid Sequence of VL CDR3 of S1scAb06
QQYAASPLT

SEQ ID NO: 11 - DNA, 351 bp, artificial sequence
  Nucleotide Sequence of VH of S1scAb11
caggtgcagctgttgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaggctacgaaaactactttgactactggggccagggaaccctggtcaccgtctcctca

SEQ ID NO: 12 - protein, 117 aa, artificial sequence
  Amino acid Sequence of VH of S1scAb11
QVQLLQSGGGVVQPGRSLRLSCAASGFTFSSYGMHWVRQAPGKGLEWVAVISYDGSNKYYADSVKGRFTISRDNSKNTLYLQMNSLRAEDTAVYYCAKGYENYFDYWGQGTLVTVSS

SEQ ID NO: 13 - protein, 8 aa, artificial sequence
  Amino acid Sequence of VH CDR1 of S1scAb11
GFTFSSYG

SEQ ID NO: 14 - protein, 8 aa, artificial sequence
  Amino acid Sequence of VH CDR2 of S1scAb11
ISYDGSNK

SEQ ID NO: 15 - protein, 10 aa, artificial sequence
  Amino acid Sequence of VH CDR3 of S1scAb11
AKGYENYFDY

SEQ ID NO: 16 - DNA, 324 bp, artificial sequence
  Nucleotide Sequence of VL of S1scAb11
gaaattgtgctgactcagtctccagacaccctgtctttgtctccaggggaaagagccaccctctcctgcagggccagtcagagtgttgccagcaactacttagcctggtaccagcagaaacctggccaggctcccacactcctcatctataatgcatccaccagggccactggcatccccgacaggttcagtggcagtgggtctgggacagacttcactctcaccatcagcagactggagcctgaagattttgcagtgtattactgtcagcagtatagtgcctcccctctcactttcggcggagggaccaaggtggagatcaaa

SEQ ID NO: 17 - protein, 108 aa, artificial sequence
  Amino acid Sequence of VL of S1scAb11
EIVLTQSPDTLSLSPGERATLSCRASQSVASNYLAWYQQKPGQAPTLLIYNASTRATGIPDRFSGSGSGTDFTLTISRLEPEDFAVYYCQQYSASPLTFGGGTKVEIK

SEQ ID NO: 18 - protein, 7 aa, artificial sequence
  Amino acid Sequence of VL CDR1 of S1scAb11
QSVASNY

SEQ ID NO: 19 - protein, 3 aa, artificial sequence
  Amino acid Sequence of VL CDR2 of S1scAb11
NAS

SEQ ID NO: 20 - protein, 9 aa, artificial sequence
  Amino acid Sequence of VL CDR3 of S1scAb11
QQYSASPLT

SEQ ID NO: 21 - DNA, 351 bp, artificial sequence
  Nucleotide Sequence of VH of S2H1
caggtgcagctgttgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgtctctttacgaaaactacttttcgctttggggccagggaaccctggtcaccgtctcctca

SEQ ID NO: 22 - protein, 117 aa, artificial sequence
  Amino acid Sequence of VH of S2H1
QVQLLQSGGGVVQPGRSLRLSCAASGFTFSSYGMHWVRQAPGKGLEWVAVISYDGSNKYYADSVKGRFTISRDNSKNTLYLQMNSLRAEDTAVYYCASLYENYFSLWGQGTLVTVSS

SEQ ID NO: 23 - protein, 8 aa, artificial sequence
  Amino acid Sequence of VH CDR1 of S2H1
GFTFSSYG

SEQ ID NO: 24 - protein, 8 aa, artificial sequence
  Amino acid Sequence of VH CDR2 of S2H1
ISYDGSNK

SEQ ID NO: 25 - protein, 10 aa, artificial sequence
  Amino acid Sequence of VH CDR3 of S2H1
ASLYENYFSL

SEQ ID NO: 26 - DNA, 324 bp, artificial sequence; identical to SEQ ID NO: 6
  Nucleotide Sequence of VL of S2H1

SEQ ID NO: 27 - protein, 108 aa, artificial sequence; identical to SEQ ID NO: 7
  Amino acid Sequence of VL of S2H1

SEQ ID NO: 28 - protein, 7 aa, artificial sequence
  Amino acid Sequence of VL CDR1 of S2H1
QSVSSNY

SEQ ID NO: 29 - protein, 3 aa, artificial sequence
  Amino acid Sequence of VL CDR2 of S2H1
GAS

SEQ ID NO: 30 - protein, 9 aa, artificial sequence
  Amino acid Sequence of VL CDR3 of S2H1
QQYAASPLT

SEQ ID NO: 31 - DNA, 351 bp, artificial sequence
  Nucleotide Sequence of VH of S2H2
caggtgcagctgttgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgacgtttcgtgaaaactactttgagtactggggccagggaaccctggtcaccgtctcctca

SEQ ID NO: 32 - protein, 117 aa, artificial sequence
  Amino acid Sequence of VH of S2H2
QVQLLQSGGGVVQPGRSLRLSCAASGFTFSSYGMHWVRQAPGKGLEWVAVISYDGSNKYYADSVKGRFTISRDNSKNTLYLQMNSLRAEDTAVYYCATFRENYFEYWGQGTLVTVSS

SEQ ID NO: 33 - protein, 8 aa, artificial sequence
  Amino acid Sequence of VH CDR1 of S2H2
GFTFSSYG

SEQ ID NO: 34 - protein, 8 aa, artificial sequence
  Amino acid Sequence of VH CDR2 of S2H2
ISYDGSNK

SEQ ID NO: 35 - protein, 10 aa, artificial sequence
  Amino acid Sequence of VH CDR3 of S2H2
ATFRENYFEY

SEQ ID NO: 36 - DNA, 324 bp, artificial sequence; identical to SEQ ID NO: 6
  Nucleotide Sequence of VL of S2H2

SEQ ID NO: 37 - protein, 108 aa, artificial sequence; identical to SEQ ID NO: 7
  Amino acid Sequence of VL of S2H2

SEQ ID NO: 38 - protein, 7 aa, artificial sequence
  Amino acid Sequence of VL CDR1 of S2H2
QSVSSNY

SEQ ID NO: 39 - protein, 3 aa, artificial sequence
  Amino acid Sequence of VL CDR2 of S2H2
GAS

SEQ ID NO: 40 - protein, 9 aa, artificial sequence
  Amino acid Sequence of VL CDR3 of S2H2
QQYAASPLT

SEQ ID NO: 41 - DNA, 351 bp, artificial sequence
  Nucleotide Sequence of VH of S2H3
caggtgcagctgttgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgggggttagggaaaactactttacttactggggccagggaaccctggtcaccgtctcctca

SEQ ID NO: 42 - protein, 117 aa, artificial sequence
  Amino acid Sequence of VH of S2H3
QVQLLQSGGGVVQPGRSLRLSCAASGFTFSSYGMHWVRQAPGKGLEWVAVISYDGSNKYYADSVKGRFTISRDNSKNTLYLQMNSLRAEDTAVYYCAGVRENYFTYWGQGTLVTVSS

SEQ ID NO: 43 - protein, 8 aa, artificial sequence
  Amino acid Sequence of VH CDR1 of S2H3
GFTFSSYG

SEQ ID NO: 44 - protein, 8 aa, artificial sequence
  Amino acid Sequence of VH CDR2 of S2H3
ISYDGSNK

SEQ ID NO: 45 - protein, 10 aa, artificial sequence
  Amino acid Sequence of VH CDR3 of S2H3
AGVRENYFTY

SEQ ID NO: 46 - DNA, 324 bp, artificial sequence; identical to SEQ ID NO: 6
  Nucleotide Sequence of VL of S2H3

SEQ ID NO: 47 - protein, 108 aa, artificial sequence; identical to SEQ ID NO: 7
  Amino acid Sequence of VL of S2H3

SEQ ID NO: 48 - protein, 7 aa, artificial sequence
  Amino acid Sequence of VL CDR1 of S2H3
QSVSSNY

SEQ ID NO: 49 - protein, 3 aa, artificial sequence
  Amino acid Sequence of VL CDR2 of S2H3
GAS

SEQ ID NO: 50 - protein, 9 aa, artificial sequence
  Amino acid Sequence of VL CDR3 of S2H3
QQYAASPLT

SEQ ID NO: 51 - DNA, 351 bp, artificial sequence
  Nucleotide Sequence of VH of S2H4
caggtgcagctgttgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgggggttagggaaaactacttttcttactggggccagggaaccctggtcaccgtctcctca

SEQ ID NO: 52 - protein, 117 aa, artificial sequence
  Amino acid Sequence of VH of S2H4
QVQLLQSGGGVVQPGRSLRLSCAASGFTFSSYGMHWVRQAPGKGLEWVAVISYDGSNKYYADSVKGRFTISRDNSKNTLYLQMNSLRAEDTAVYYCAGVRENYFSYWGQGTLVTVSS

SEQ ID NO: 53 - protein, 8 aa, artificial sequence
  Amino acid Sequence of VH CDR1 of S2H4
GFTFSSYG

SEQ ID NO: 54 - protein, 8 aa, artificial sequence
  Amino acid Sequence of VH CDR2 of S2H4
ISYDGSNK

SEQ ID NO: 55 - protein, 10 aa, artificial sequence
  Amino acid Sequence of VH CDR3 of S2H4
AGVRENYFSY

SEQ ID NO: 56 - DNA, 324 bp, artificial sequence; identical to SEQ ID NO: 6
  Nucleotide Sequence of VL of S2H4

SEQ ID NO: 57 - protein, 108 aa, artificial sequence; identical to SEQ ID NO: 7
  Amino acid Sequence of VL of S2H4

SEQ ID NO: 58 - protein, 7 aa, artificial sequence
  Amino acid Sequence of VL CDR1 of S2H4
QSVSSNY

SEQ ID NO: 59 - protein, 3 aa, artificial sequence
  Amino acid Sequence of VL CDR2 of S2H4
GAS

SEQ ID NO: 60 - protein, 9 aa, artificial sequence
  Amino acid Sequence of VL CDR3 of S2H4
QQYAASPLT

SEQ ID NO: 61 - DNA, 351 bp, artificial sequence
  Nucleotide Sequence of VH of S2H5
caggtgcagctgttgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgtctcgttacgaaaactacttttctctgtggggccagggaaccctggtcaccgtctcctca

SEQ ID NO: 62 - protein, 117 aa, artificial sequence
  Amino acid Sequence of VH of S2H5
QVQLLQSGGGVVQPGRSLRLSCAASGFTFSSYGMHWVRQAPGKGLEWVAVISYDGSNKYYADSVKGRFTISRDNSKNTLYLQMNSLRAEDTAVYYCASRYENYFSLWGQGTLVTVSS

SEQ ID NO: 63 - protein, 8 aa, artificial sequence
  Amino acid Sequence of VH CDR1 of S2H5
GFTFSSYG

SEQ ID NO: 64 - protein, 8 aa, artificial sequence
  Amino acid Sequence of VH CDR2 of S2H5
ISYDGSNK

SEQ ID NO: 65 - protein, 10 aa, artificial sequence
  Amino acid Sequence of VH CDR3 of S2H5
ASRYENYFSL

SEQ ID NO: 66 - DNA, 324 bp, artificial sequence; identical to SEQ ID NO: 6
  Nucleotide Sequence of VL of S2H5

SEQ ID NO: 67 - protein, 108 aa, artificial sequence; identical to SEQ ID NO: 7
  Amino acid Sequence of VL of S2H5

SEQ ID NO: 68 - protein, 7 aa, artificial sequence
  Amino acid Sequence of VL CDR1 of S2H5
QSVSSNY

SEQ ID NO: 69 - protein, 3 aa, artificial sequence
  Amino acid Sequence of VL CDR2 of S2H5
GAS

SEQ ID NO: 70 - protein, 9 aa, artificial sequence
  Amino acid Sequence of VL CDR3 of S2H5
QQYAASPLT

SEQ ID NO: 71 - DNA, 351 bp, artificial sequence
  Nucleotide Sequence of VH of S2H6
caggtgcagctgttgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgtcttttcaggaaaactactttacgtactggggccagggaaccctggtcaccgtctcctca

SEQ ID NO: 72 - protein, 117 aa, artificial sequence
  Amino acid Sequence of VH of S2H6
QVQLLQSGGGVVQPGRSLRLSCAASGFTFSSYGMHWVRQAPGKGLEWVAVISYDGSNKYYADSVKGRFTISRDNSKNTLYLQMNSLRAEDTAVYYCASFQENYFTYWGQGTLVTVSS

SEQ ID NO: 73 - protein, 8 aa, artificial sequence
  Amino acid Sequence of VH CDR1 of S2H6
GFTFSSYG

SEQ ID NO: 74 - protein, 8 aa, artificial sequence
  Amino acid Sequence of VH CDR2 of S2H6
ISYDGSNK

SEQ ID NO: 75 - protein, 10 aa, artificial sequence
  Amino acid Sequence of VH CDR3 of S2H6
ASFQENYFTY

SEQ ID NO: 76 - DNA, 324 bp, artificial sequence; identical to SEQ ID NO: 6
  Nucleotide Sequence of VL of S2H6

SEQ ID NO: 77 - protein, 108 aa, artificial sequence; identical to SEQ ID NO: 7
  Amino acid Sequence of VL of S2H6

SEQ ID NO: 78 - protein, 7 aa, artificial sequence
  Amino acid Sequence of VL CDR1 of S2H6
QSVSSNY

SEQ ID NO: 79 - protein, 3 aa, artificial sequence
  Amino acid Sequence of VL CDR2 of S2H6
GAS

SEQ ID NO: 80 - protein, 9 aa, artificial sequence
  Amino acid Sequence of VL CDR3 of S2H6
QQYAASPLT

SEQ ID NO: 81 - DNA, 351 bp, artificial sequence
  Nucleotide Sequence of VH of S2H7
caggtgcagctgttgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgactcggtacgaaaactacttttctacgtggggccagggaaccctggtcaccgtctcctca

SEQ ID NO: 82 - protein, 117 aa, artificial sequence
  Amino acid Sequence of VH of S2H7
QVQLLQSGGGVVQPGRSLRLSCAASGFTFSSYGMHWVRQAPGKGLEWVAVISYDGSNKYYADSVKGRFTISRDNSKNTLYLQMNSLRAEDTAVYYCATRYENYFSTWGQGTLVTVSS

SEQ ID NO: 83 - protein, 8 aa, artificial sequence
  Amino acid Sequence of VH CDR1 of S2H7
GFTFSSYG

SEQ ID NO: 84 - protein, 8 aa, artificial sequence
  Amino acid Sequence of VH CDR2 of S2H7
ISYDGSNK

SEQ ID NO: 85 - protein, 10 aa, artificial sequence
  Amino acid Sequence of VH CDR3 of S2H7
ATRYENYFST

SEQ ID NO: 86 - DNA, 324 bp, artificial sequence; identical to SEQ ID NO: 6
  Nucleotide Sequence of VL of S2H7

SEQ ID NO: 87 - protein, 108 aa, artificial sequence; identical to SEQ ID NO: 7
  Amino acid Sequence of VL of S2H7

SEQ ID NO: 88 - protein, 7 aa, artificial sequence
  Amino acid Sequence of VL CDR1 of S2H7
QSVSSNY

SEQ ID NO: 89 - protein, 3 aa, artificial sequence
  Amino acid Sequence of VL CDR2 of S2H7
GAS

SEQ ID NO: 90 - protein, 9 aa, artificial sequence
  Amino acid Sequence of VL CDR3 of S2H7
QQYAASPLT

SEQ ID NO: 91 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 92 - protein, 330 aa, Homo sapiens [sequence omitted]

SEQ ID NO: 93 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 94 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 95 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 96 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 98 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 99 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 9 aa, artificial sequence
  Peptide 01
AVQVRCKRL

SEQ ID NO: 101 - protein, 18 aa, artificial sequence
  Peptide 02
DAKSKSVSLPVPDLCAVY

SEQ ID NO: 102 - protein, 12 aa, artificial sequence
  Peptide 03
EKPVFPENNLQF

SEQ ID NO: 103 - protein, 100 aa, artificial sequence
  Subdomain NTD F22-Q121 of the LEPR Extracellular Domain
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQ

SEQ ID NO: 104 - protein, 212 aa, artificial sequence
  Subdomain CRH1 Q122-V333  of the LEPR Extracellular Domain
QIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDV

SEQ ID NO: 105 - protein, 94 aa, artificial sequence
  Subdomain IgD I334-V427 of the LEPR Extracellular Domain
IYFPPKILTSVGSNVSFHCIYKKENKIVPSKEIVWWMNLAEKIPQSQYDVVSDHVSKVTFFNLNETKPRGKFTYDAVYCCNEHECHHRYAELYV

SEQ ID NO: 106 - protein, 208 aa, artificial sequence
  Subdomain CRH2 I428-D635 of the LEPR Extracellular Domain
IDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMD

SEQ ID NO: 107 - protein, 204 aa, artificial sequence
  Subdomain FNIII I636-D839 of the LEPR Extracellular Domain
IKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSD